SEQUENCE LISTING (43 sequences)

SEQ ID NO: 1 - DNA, 1149 bp, unidentified
  source 1..1149: mol_type = other DNA
agattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgattctcc

SEQ ID NO: 2 - DNA, 1151 bp, unidentified
  source 1..1151: mol_type = other DNA
cgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgattct

SEQ ID NO: 3 - DNA, 1149 bp, unidentified
  source 1..1149: mol_type = other DNA
cagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgattctc

SEQ ID NO: 4 - DNA, 1152 bp, unidentified
  source 1..1152: mol_type = other DNA
cgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgattctc

SEQ ID NO: 5 - DNA, 1146 bp, unidentified
  source 1..1146: mol_type = other DNA
cagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccgggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatacatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgatt

SEQ ID NO: 6 - DNA, 1151 bp, unidentified
  source 1..1151: mol_type = other DNA
gcgtcagattgcagaaaagagtattatcactagctccaaacacaaacttgaacactgcaggtcagtcaattctcaatgattacaatgccatagcatccagagtgaatgggaagacttatgctcttttggaccaaacggcaatattatccccttacactatacatgcaccaataatttcactggccgttagaatatctactgatgattatgatgacatgagaaatggagttgagtctatactagactgtttggctgcggcaattcgtactgaaggctcgagaccggttagagtgattgaacgtagggttattgaaccagtggtaaaacagctggtcgaagatctgaagttgaaaagtctagtttctgaaatatcaattgccaatttcgctgctgccgatactgcgcttattcaaccagagatagtagaaactgaaaatccattaatagttggtatcatagaacaggtggttgtaagacaaccagccagtctaaatggtggtaatattagagcagcgattggcaggtggtcaggtaataaaggctcagtcacatgtgtctcaggcatggaagcagaacatatgttttttgtggaactaaaagctagaacgtgtggtgtgctgaacgtcgtttatctgccagctccaggagttataatggtgcctatgccgcaaggacgcaacagagaaagtgttatacttgacgtatccgcagagatgacagcagatgattttataattgatttctttgatgacaacaacattgttcatacggaaagaggagttggcctattttcatttccaatgtgtaccagaattaggtttagagttacaccatggacacagcaaaaatctcagaatggacttgacactccatcattggctacgtgggcgaacggcacatctccgagacagccagcggtgtctttcatgtttgaattaagaagaaccttcactgaaaatgattataaattcgtttcaagatgtacctcaaaagttcaatacatattggataccaacttcccagagacatcttttattaacaggcctcaaatagaatggaacgtacaagagatgattacttctgacactgatacagtatggtcacgtaaaatcgcaatgctagtcgcagcatttgctgctaagatctgattc

SEQ ID NO: 7 - DNA, 1146 bp, unidentified; identical to SEQ ID NO: 5
  source 1..1146: mol_type = other DNA

SEQ ID NO: 8 - DNA, 1154 bp, unidentified
  source 1..1154: mol_type = other DNA
gcgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgattctcc

SEQ ID NO: 9 - DNA, 1146 bp, unidentified; identical to SEQ ID NO: 5
  source 1..1146: mol_type = other DNA

SEQ ID NO: 10 - DNA, 1147 bp, unidentified
  source 1..1147: mol_type = other DNA
cgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctga

SEQ ID NO: 11 - DNA, 1154 bp, unidentified; identical to SEQ ID NO: 8
  source 1..1154: mol_type = other DNA

SEQ ID NO: 12 - DNA, 1162 bp, unidentified
  source 1..1162: mol_type = other DNA
gtgaacgcttgcgtcagattgcagaaaagagtattatcactagctccaaacacaaacttgaacactgcaggtcagtcaattctcaatgattacaatgccatagcatccagagtgaatgggaagacttatgctcttttggaccaaacggcaatattatccccttacactatacatgcaccaataatttcactggccgttagaatatctactgatgattatgatgacatgagaaatggagttgagtctatactagactgtttggctgcggcaattcgtactgaaggctcgagaccggttagagtgattgagcgtagggttattgaaccagtggtaaaacagctggtcgaagatctgaagttgaaaagtctagtttctgaaatatcaattgccaatttcgctgctgccgatactgcgcttattcaaccagagatagtagaaactgaaaatccattaatagttggtatcatagaacaggtggttgtaagacaaccagccagtctaaatggtggtaatattagagcagcgattggcaggtggtcaggaataaaggctcagtcacatgtgtctcaggcatggaagcagaacatatgttttttgtggaactaaaagctagaacgtgtggtgtgctgaacgtcgtttatctgccagctccaggagttataatggtgcctatgccgcaaggacgcaacagagaaagtgttatacttgacgtatccgcagagatgacagcagatgattttataattgatttctttgatgacaacaacattgttcatacggaaagaggagttggcctattttcatttccaatgtgtaccagaattaggtttagagttacaccatggacacagcaaaaatctcagaatggacttgacactccatcattggctacgtgggcgaacggcacatctccgagacagccagcggtgtctttcatgtttgaattaagaagaaccttcactgaaaatgattacaaattcgtttcaagatgtacctcaaaagttcaatacatattggataccaacttcccagagacatcttttattaacaggcctcaaatagaatggaacgtacaagagatgattacttctgacactgatacagtatggtcacgtaaaatcgcaatgctagtcgcagcatttgctgctaagatctgattctc

SEQ ID NO: 13 - DNA, 1149 bp, unidentified; identical to SEQ ID NO: 1
  source 1..1149: mol_type = other DNA

SEQ ID NO: 14 - DNA, 1149 bp, unidentified
  source 1..1149: mol_type = other DNA
tgcgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctga

SEQ ID NO: 15 - DNA, 1152 bp, unidentified
  source 1..1152: mol_type = other DNA
tgcgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatatatattggataccaacttcccagagacatcatttattaacaggcctcaaatagaatggaacgtacaagagatgattacttctgacacagacacagtatggtcacgtaaaatcgcaatgctagtcgcagcatttgctgctaagatctgattc

SEQ ID NO: 16 - DNA, 1221 bp, unidentified
  source 1..1221: mol_type = other DNA
gtgtacgagcatggatctgatcgaaacggtgaacgcttgcgtcagattgcagaaaagagtattatcactagctccaaacacaaacttgaacactgcaggtcagtcaattctcaatgattataatgctatagcatccagagtgaatgggaagacttatgctcttttggaccaaacagcaatattatccccttacacgatacacgcaccgataatttcgctggccgttagaatatctactgatgattatgatgacatgagaaatggagttgagtctatactagattgtttggctgcggcgattcgcactgaaggctcgagaccggttagagtgattgaacgtagagttattgaaccagtggtaaagcagctggtcgaagatctgaagttaaaaagtctgatttctgaaatctcaattgccaatttcgctgctactgataccgcgcttatccaaccagaagtagtagaaactgaaaatccattgatagttggtatcatagaacaggtggttgtaagacaaccagccagtctaaatggtggcaatattagagcagcgattggcagatggtcaggtaataaaggctcagtcacatgtgtctcaggcatggaagcagaacatatgttcttcgtggaactaaaagctaggacgtgtggtgtactgaacgtcgtttatctgccagccccaggagttataatggtgcctatgccgcaaggacgcaacagagaaagtgttatacttgacgtatccgcagagatgacagcagatgattttataatcgatttctttgatgataacaacattgtccatacggaaagaggagttggcctattttcatttccaatgtgtaccagaattagatttagagttacaccatggacacaacaaaaatctcagaatggacttgacactccatcattggctacgtgggcgaacggtacgtctccgaggcagccagcggtgtctttcatgtttgaattaagaagaaccttcactgaaaacgattataaattcgtttcacgatgtacctcgaaagttcaatacatattggagaccaacttcccagagacatcatttattaacaggcctcaaatagaatggaacgtacaagagatgattacttctgacacagacacagtatggtcacgtaaaatcgcaatgctagtcgcagcatttgctgctaagatctgattctccctgagcccgggagccgggttgctctagag

SEQ ID NO: 17 - protein, 391 aa, unidentified
MDLIETVNACVRLQKRVLSLAPNTNLNTAGQSILNDYNAIASRVNGKTYALLDQTAILSPYTIHAPIISLAVRISTDDYDDMRNGVESILDCLAAAIRTEGSRPVRVIERRVIEPVVKQLVEDLKLKSLISEISIANFAATDTALIQPEVVETENPLIVGIIEQVVVRQPASLNGGNIRAAIGRWSGNKGSVTCVSGMEAEHMFFVELKARTCGVLNVVYLPAPGVIMVPMPQGRNRESVILDVSAEMTADDFIIDFFDDNNIVHTERGVGLFSFPMCTRIRFRVTPWTQQKSQNGLDTPSLATWANGTSPRQPAVSFMFELRRTFTENDYKFVSRCTSKVQYILETNFPETSFINRPQIEWNVQEMITSDTDTVWSRKIAMLVAAFAAKI

SEQ ID NO: 18 - DNA, 20 bp, synthetic construct
  source 1..20: mol_type = other DNA
ggctttaaaagcgagaattt

SEQ ID NO: 19 - DNA, 19 bp, synthetic construct
  source 1..19: mol_type = other DNA
ggtcacatcatacaactct

SEQ ID NO: 20 - DNA, 20 bp, synthetic construct
  source 1..20: mol_type = other DNA
ccagtaggccatcctttagt

SEQ ID NO: 21 - DNA, 25 bp, synthetic construct
  source 1..25: mol_type = other DNA
gtccacttaatacacaaactctagg

SEQ ID NO: 22 - DNA, 25 bp, synthetic construct
  source 1..25: mol_type = other DNA
gcagtrtccattgaaccagtaattg

SEQ ID NO: 23 - DNA, 24 bp, synthetic construct
  source 1..24: mol_type = other DNA
gayataacggctgatccaactacg

SEQ ID NO: 24 - DNA, 21 bp, synthetic construct
  source 1..21: mol_type = other DNA
ctccacagattggacgaatga

SEQ ID NO: 25 - DNA, 20 bp, synthetic construct
  source 1..20: mol_type = other DNA
gtgaacgcttgcgtcagatt

SEQ ID NO: 26 - DNA, 20 bp, synthetic construct
  source 1..20: mol_type = other DNA
cgggctcagggagaatcaga

SEQ ID NO: 27 - DNA, 20 bp, synthetic construct
  source 1..20: mol_type = other DNA
ccgcagagatgacagcagat

SEQ ID NO: 28 - DNA, 20 bp, synthetic construct
  source 1..20: mol_type = other DNA
ctggttggataagcgcggta

SEQ ID NO: 29 - DNA, 20 bp, synthetic construct
  source 1..20: mol_type = other DNA
accatctwcacrtraccctc

SEQ ID NO: 30 - DNA, 20 bp, synthetic construct
  source 1..20: mol_type = other DNA
ggtcacataacgcccctata

SEQ ID NO: 31 - DNA, 33 bp, synthetic construct
  source 1..33: mol_type = other DNA
atgagcacaatagttaaaagctaacactgtcaa

SEQ ID NO: 32 - DNA, 22 bp, synthetic construct
  source 1..22: mol_type = other DNA
gatgttaccacgaccacttgta

SEQ ID NO: 33 - DNA, 19 bp, synthetic construct
  source 1..19: mol_type = other DNA
agttggatcggccgttatg

SEQ ID NO: 34 - DNA, 25 bp, synthetic construct
  source 1..25: mol_type = other DNA
tgggaccacgagagaatgtagctgt

SEQ ID NO: 35 - DNA, 17 bp, synthetic construct
  source 1..17: mol_type = other DNA
atccgactacggctcca

SEQ ID NO: 36 - DNA, 22 bp, synthetic construct
  source 1..22: mol_type = other DNA
tgcagcagaatttaatgatcgc

SEQ ID NO: 37 - DNA, 22 bp, synthetic construct
  source 1..22: mol_type = other DNA
tgcagcagaatttaatgatcgc

SEQ ID NO: 38 - DNA, 22 bp, synthetic construct
  source 1..22: mol_type = other DNA
catccagagtgaatgggaagac

SEQ ID NO: 39 - DNA, 21 bp, synthetic construct
  source 1..21: mol_type = other DNA
ttctaacggccagcgaaatta

SEQ ID NO: 40 - DNA, 23 bp, synthetic construct
  source 1..23: mol_type = other DNA
cccttacacgatacacgcaccga

SEQ ID NO: 41 - DNA, 23 bp, synthetic construct
  source 1..23: mol_type = other DNA
gccttctgattctacgtcaacta

SEQ ID NO: 42 - DNA, 24 bp, synthetic construct
  source 1..24: mol_type = other DNA
cttgttgtacgcttcttcgtattc

SEQ ID NO: 43 - DNA, 24 bp, synthetic construct
  source 1..24: mol_type = other DNA
aacatcaagtcgtagcgacgcagt

(19) United States
(12) Patent Application Publication
(10) Pub. No.: US 2024/0018610 A1
(43) Pub. Date: Jan. 18, 2024

(54) Multiplex Assay for Simultaneous Detection of Equine Group A and B Rotaviruses and Genotyping of Equine Rotavirus A G3 And G14

(71) Applicant: Board of Supervisors of Louisiana State University and Agricultural and Mechanical College, Baton Rouge, LA (US)

(72) Inventors: Mariano Carossino, Baton Rouge, LA (US); Udeni B.R. Balasuriya, Baton Rouge, LA (US)

(21) Appl. No.: 18/350,071

(22) Filed: Jul. 11, 2023

Related U.S. Application Data
(60) Provisional application No. 63/389,216, filed on Jul. 14, 2022.

(51) Int. Cl. C12Q 1/70

(52) U.S. Cl. CPC C12Q 1/701; C12Q 2600/16; C12Q 2600/156

(57) ABSTRACT

Embodiments of the present disclosure provide for embodiments of a panel of oligonucleotides for use in a multiplex reverse transcriptase-polymerase chain reaction (RT-PCR) assay for the identification of rotavirus A and B and genotypes thereof. Another aspect of the disclosure encompasses embodiments of an RT-PCR multiplex method for determining whether an equine is infected with an equine rotavirus.

### CROSS-REFERENCE TO RELATED APPLICATIONS

This application claims benefit of U.S. Provisional Application No. 63/389,216, filed Jul. 14, 2022, which is hereby incorporated herein by reference in its entirety.

### SEQUENCE LISTING

This application contains a sequence listing filed in ST.26 format entitled “221205-1590 Sequence Listing” created on Jul. 3, 2023, having 57,491 bytes. The content of the sequence listing is incorporated herein in its entirety.

### BACKGROUND

Equine rotavirus A (ERVA) is the leading cause of diarrhea in neonatal foals, with a significant impact on the equine breeding industry. The most prevalent ERVA genotypes infecting foals include G3P[12] and G14P[12], with other genomic arrangements being rare. Recently, a group B equine rotavirus (ERVB) has been identified as a cause of foal diarrhea in the US, thus, demonstrating an urgent need for molecular diagnostics for the detection and understanding of its molecular epidemiology. A one-step 3-plex TaqMan® real-time reverse transcription polymerase chain reaction (RT-qPCR) assay was previously developed for rapid detection and G-typing of ERVA directly from fecal specimens. With the emergence of ERVB and the immediate need to better understand its impact and inform the need for vaccine development, incorporation into routine diagnostic assays is imperative.

### SUMMARY

Embodiments of the present disclosure provide for embodiments of a panel of oligonucleotides for use in a multiplex reverse transcriptase-polymerase chain reaction (RT-PCR) assay for the identification of rotavirus A and B and genotypes thereof, the panel of nucleotides comprising: a rotavirus A-specific forward PCR primer, a rotavirus A-specific reverse PCR primer, and a labeled oligonucleotide probe, wherein the forward PCR primer, reverse PCR primer, and the labeled oligonucleotide probe are each complementary to a region of a nucleic acid, or the complement thereof, encoding the non-structural protein 3 (NSP3) of an equine rotavirus A; a rotavirus A VP7 (subtype G3 genotype)-specific forward PCR primer, a rotavirus A VP7 (subtype G3 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; a rotavirus A VP7 (subtype G14 genotype)-specific forward PCR primer, a rotavirus A VP7 (subtype G14 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; and either (i) a rotavirus B VP6-specific forward PCR primer, a rotavirus B VP6-specific reverse PCR primer, and a labeled oligonucleotide, wherein the forward PCR primer, reverse PCR primer, and the labeled oligonucleotide probe are each complementary to a nucleic acid, or the complement thereof, encoding the structural protein VP6, or a fragment thereof, of an equine rotavirus B, or (ii) a rotavirus B non-structural protein 5 (NSP5)-specific forward PCR primer, a rotavirus B an NSP5-specific reverse PCR primer, and a labeled oligonucleotide probe, wherein the forward PCR primer, reverse PCR primer, and the labeled oligonucleotide probe are each complementary to a nucleic acid, or the complement thereof, encoding the non-structural protein NSP5, or a fragment thereof, of an equine rotavirus B.

Another aspect of the disclosure encompasses embodiments of an RT-PCR multiplex method for determining whether an equine is infected with an equine rotavirus, the method comprising the steps of: (a) obtaining a fecal sample from the equine; (b) extracting RNA from the fecal sample; (c) assaying the RNA by an RT-PCR multiplex assay performed with a panel of oligonucleotides comprising: a rotavirus A-specific forward PCR primer, a rotavirus A-specific reverse PCR primer, and a labeled oligonucleotide probe, wherein the forward PCR primer, reverse PCR primer, and the labeled oligonucleotide probe are each complementary to a region of a nucleic acid, or the complement thereof, encoding the non-structural protein 3 (NSP3) of an equine rotavirus A; a rotavirus A VP7 (subtype G3 genotype)-specific forward PCR primer, a rotavirus A VP7 (subtype G3 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; a rotavirus A VP7 (subtype G14 genotype)-specific forward PCR primer, a rotavirus A VP7 (subtype G14 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; and either (i) a rotavirus B VP6-specific forward PCR primer, a rotavirus B VP6-specific reverse PCR primer, and a labeled oligonucleotide, wherein the forward PCR primer, reverse PCR primer, and the labeled oligonucleotide probe are each complementary to a nucleic acid, or the complement thereof, encoding the structural protein VP6, or a fragment thereof, of an equine rotavirus B, or (ii) a rotavirus B non-structural protein 5 (NSP5)-specific forward PCR primer, a rotavirus B an NSP5-specific reverse PCR primer, and a labeled oligonucleotide probe, wherein the forward PCR primer, reverse PCR primer, and the labeled oligonucleotide probe are each complementary to a nucleic acid, or the complement thereof, encoding the non-structural protein NSP5, or a fragment thereof, of an equine rotavirus B, wherein detection of the rotavirus A or B indicates an infection of the equine with the rotavirus A or B and whether, if present, the rotavirus A is of the genotype VP7 subtype G3 or G14.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 Negative staining of equine rotavirus B (ERVB) particles in fecal specimens. ERVB particle size ranged from 48.2 to 62.4 nm and showed a typical “wheel” shape with spike-like projections from the outer capsid. Transmission electron microscopy, 40,000× magnification. Bar=50 μm.

FIG. 2 Comparison of the analytical sensitivity of the ERVA/ERVB VP6 and ERVA/ERVB NSP5 4-plex RT-qPCR assays. Ct, cycle threshold; IVT RNA, in vitro transcribed RNA.

FIG. 3 Nucleotide sequence alignment between the reference ERVB VP6 gene sequence (GenBank Accession Number MZ327693.1, labeled as 1, SEQ ID NO:16 (partial)) and RVB/Horse-wt/USA/KY1-6/2021 and RVB/Horse-wt/USA/KY1-13/2021 (labeled as 2, SEQ ID NO:12 (partial)). The alignment window is limited to the region targeted by the ERVB VP6-specific RT-qPCR assay (nt 132-230). ERVB VP6-specific primer and probe nucleotide sequences (ERVB-VP6-F (SEQ ID NO:38), ERVB-VP6-R (SEQ ID NO: 39) and ERVB-VP6-P (SEQ ID NO:40)) are mapped. Nucleotide substitutions are marked with boxes.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Abbreviations

RVA: rotavirus A; ERVA: equine rotavirus A; ERVB: equine rotavirus B; BRVA: bovine rotavirus A; SRVA: simian rotavirus A; RNA: ribonucleic acid; DNA: deoxyribonucleic acid; RT-qPCR: reverse transcription real-time polymerase chain reaction; dNTP: deoxynucleotide triphosphate; LOD: limit of detection; TCF: tissue culture fluid; EMEM: Eagle's minimum essential medium; IVT: in vitro transcribed.

### Definitions

The term “Rotaviruses” as used herein refers to icosahedral, non-enveloped viruses with a double-stranded, segmented RNA genome (dsRNA) that belong to the family Sedoreoviridae (genus Rotavirus) [8, 9]. The ERVA genome consists of 11 double-stranded RNA segments, five of which encode for the viral structural proteins (VP1-4, 6, and 7) while the remaining segments encode for non-structural proteins (NSP1-6).

The structural proteins assemble into a triple capsid: the outer capsid composed of VP7 and VP4, the intermediate integrated by VP6 and the inner capsid formed by VP1, VP2, and VP3 [10-12]. The VP6 is used to classify rotaviruses in groups (A-H), among which group A is the most common cause of diarrhea in humans and animals [13]. The two outer capsid proteins, VP7 and VP4, are the most variable and immunogenic proteins of the virus, which independently elicit neutralizing antibodies following infection [1, 14]; VP7 is considered to have the major neutralizing epitopes of the virus.

Group A rotaviruses are further classified into G-types and P-types according to the nucleotide identity of these two proteins, respectively (19). While seven G-types (G3, G5, G6, G8, G10, G13, and G14) and six P-types (P[1], P[3], P[7], P[11], P[12], and P[18]) have been identified among ERVA strains, the G3P[12] and G14P[12] genotypes are the most prevalent and epidemiologically relevant in the US and elsewhere [1, 2, 15-17]. In recent years, novel “equine-like” G3 strains have been identified in children around the world, demonstrating that reassortants derived from rotaviruses affecting animal species can emerge, jump species (zoonosis), and consequently have a public health impact [18-21]. It has been determined that ERVA G14 strains are the most prevalent in Central Kentucky [22].”

### DISCUSSION

Provided is a 4-plex TaqMan® RT-qPCR assay for differentiation of ERVA and ERVB and simultaneous G-typing of ERVA strains. The present disclosure, therefore, encompasses embodiments of a one-step 4-plex TaqMan® RT-qPCR assays targeting the NSP3 and VP7 genes of the ERVA G3 and G14 genotypes, as well as either the VP6 gene or the NSP5 gene of ERVB (ERVA/ERVB VP6 and ERVA/ERVB NSP5, respectively). The analytical performance was compared to a previously established ERVA 3-plex RT-qPCR assay and the clinical performance was evaluated using a panel of 193 archived fecal samples and compared to an ERVA VP7-specific or ERVB VP6-specific standard RT-PCR assay and Sanger sequencing.

The two 4-plex (multiplex) assays incorporating ERVB VP6 or NSP5 gene targets were developed. The ERVA/ERVB VP6-based 4-plex RT-qPCR assay demonstrated high sensitivity/specificity for every target (sensitivity>90% and specificity of 100%) and high overall agreement (>96%) with the result from conventional RT-PCR and sequencing. Comparison between the previous 3-plex and the 4-plex assay of the disclosure revealed only a slightly higher sensitivity for the ERVA NSP3 target using the 3-plex format (p-value 0.008) while no significant differences were detected in the sensitivity and specificity of other targets. Thus, the novel ERVA/ERVB VP6-based 4-plex RT-qPCR assay of the present disclosure advantageously provides a method for the rapid surveillance of both ERVA and ERVB circulating strains.

Equine rotavirus A (ERVA) is the leading cause of diarrhea in neonatal foals up to 3 months of age, being responsible for 20 to 77% of foal diarrhea cases in many countries and with disease outbreaks occurring on every foaling season. The high fecal viral shedding (reaching up to 109 virus particles per gram of feces) from infected foals and the high resistance in the environment contribute to their rapid transmission to susceptible foals and environmental persistence [1-7]. ERVA infection in young foals has a high morbidity rate, causing life-threatening watery diarrhea, and, thus, is considered a major health problem to the equine breeding industry, causing significant economic losses worldwide.

Control of ERVA infection in young foals is achieved by the routine vaccination of mares with an inactivated vaccine which only provides partial protection in foals and strict husbandry/hygienic practices to reduce the viral burden in the environment [1, 7, 23-25]. Variability in the level of protection depends on several factors related to the host, the circulating strain(s) of the virus, and the environment. Antigenic variation among ERVA genotypes has been shown to compromise vaccine efficacy [22, 26-30]. Also, the temporal and spatial variations in circulating ERVA strains can potentially have an impact on year-to-year variations in the efficacy of vaccination practices [2, 22, 31]. Therefore, it is important to perform genotypic characterization of ERAV strains in order to understand the molecular epidemiology of ERVA, identify novel viral reassortants and potential interspecies transmission, and assess vaccine performance in the field.

An equine group B rotavirus (ERVB) has also been identified as the etiological agent involved in localized outbreaks of diarrhea in Central Kentucky [32]. Singleplex RT-qPCR assays were developed [32]; however, there is an on-going need for cost-effective molecular-based assays that can simultaneously differentiate ERVA from ERVB to strengthen surveillance efforts.

A one-step multiplex (3-plex) TaqMan® RT-qPCR assay that allows both detection and genotypification of ERVA in a single reaction by targeting the NSP3, G3 VP7, and G14 VP7 genes of ERVA was previously developed and validated. It has now been found advantageous to develop a 4-plex RT-qPCR assay for the differentiation of ERVA and ERVB, and the simultaneous genotypification of ERVA in feces. This new multiplex RT-qPCR assay has a performance nearly equivalent to the previously developed assay, as well as with conventional ERVA VP7-specific and ERVB VP6-specific RT-PCR and Sanger sequencing.

Synthesis of ERVA and ERVB in vitro transcribed RNA for analytical performance evaluation: For ERVA, a previously synthesized in vitro transcribed (IVT) RNA with a 493 nt insert containing the targeted regions (NSP3, G3 VP7 and G14 VP7) was prepared and used as described [38]. A similar approach was used to develop ERVB IVT RNA containing the target sequences. Briefly, a 214 bp insert containing the target regions (VP6 [nt position 132-230] and NSP5 [nt position 124-238] from ERVB strain Rotavirus B isolate RVB/Horse-wt/USA/KY1518/2021 (GenBank Accession numbers KM454497.1 and KM454508.1, respectively) was cloned into the pGEM-3Z vector (Promega, Madison, WI) downstream of the T7 promoter (pERVBVP6NSP5) by GeneArt® Gene Synthesis (ThermoFisher Scientific, Regensburg, Germany). Transformed E. coli K12 DH10B™ T1R were cultured overnight at 37° C. with shaking (270 rpm), plasmid DNA was purified, linearized with HindIII and subjected to in vitro transcription using the Megascript® T7 Transcription kit (ThermoFisher Scientific, Waltham, MA) followed by DNase treatment and purification as we previously described in detail [38]. The number of ERVA and ERVB IVT RNA molecules per microliter (copies/μl) was calculated according to the following formula:

$$Number of I V T R N A molecules / μL = \frac{Avogadro ' s number ( 6.022 \times 1 {0}^{23} ) \times I V T R N A concentration ( g / μL )}{I V T R N A molecular weight ( g )}$$

The concentration of IVT RNA was adjusted to 10⁷ copies/μl using nuclease-free water containing 40 ng/μl of Ambion Yeast tRNA (ThermoFisher Scientific), and serially ten-fold diluted (10⁷-1 IVT RNA copies/μl) using nuclease-free water containing Ambion Yeast tRNA.

ERVA and ERVB-specific multiplex TaqMan® real-time RT-PCR assays targeting G3 VP7, G14 VP7 and NSP3 genes of ERVA, and VP6 or NSP5 genes of ERVB. The G3 VP7, G14 VP7 and NSP3-specific assays were multiplexed as previously described [38]. In addition, ERVB VP6- or ERVB NSP5-specific primers and probes (Table 2) were included to generate two different 4-plex assays (namely, ERVA/ERVB VP6 or ERVA/ERVB NSP5 4-plex). The reaction was set up using the QuantiTect® Multiplex RT-PCR kit (Qiagen) in a 25 μl reaction containing 12.5 μl of 2× QuantiTect Multiplex RT-PCR Master Mix with ROX, 0.25 μl QuantiTect RT Mix, 200 nM of each TaqMan® fluorogenic probe, 200 nM of each primer, and 5 μl of template RNA previously denatured at 95° C. for 5 min. An ABI 7500 Fast Real-time PCR System (Applied Biosystems®) was used with the following program: 20 min at 50° C. (reverse transcription step), 15 min at 95° C. (PCR initial activation step), 40 cycles at 94° C. for 45 sec (denaturation) and 60° C. for 75 sec (combined annealing/extension).

Statistical analysis. ERVA or ERVB IVT RNA (10⁷ to 1 IVT RNA copies/μl) were used to generate standard curves. For analytical performance, regression analysis and coefficients of determination (R²) were used to assess curve fitness and PCR amplification efficiencies (%) were calculated using the formula:

$$E = [ 1 {0}^{- \frac{1}{slope}} - 1 ] \times 100 .$$

Limit of detection with 95% confidence (LOD_{95%}) was determined by probit analysis (a non-linear regression model) using IBM SPSS Statistics (Chicago, IL, USA) where possible with 12 replicates per dilution near the detection limit (104-10 IVT RNA copies/μl). Precision (within-run and between-run imprecision) of the ERVA/ERVB VP6 or ERVA/ERVB NSP5 4-plex assays was determined as previously described (Carossino et al., 2019) with 12 replicates on the same run (within-run imprecision) or three replicates tested on two different operational days. The coefficient of variation (CV %) was determined for each target (VP6, NSP5, NSP3, G3 and G14). Cycle threshold (Ct) cut-off values were determined as the average Ct+3 standard deviations of 12 replicates of the endpoint dilution [39].

Clinical performance of the ERVA/ERVB-VP6 4-plex RT-qPCR assay was evaluated in fecal specimens and compared to the ERVA VP7-specific RT-PCR, ERVB VP6-specific RT-PCR, and G-typing by Sanger sequencing as well as previously recorded results from the ERVA 3-plex RT-qPCR assay [38]. Contingency tables (2×2) were generated to determine the sensitivity, specificity, and agreement (kappa statistic) of each target within the ERVA/ERVB-VP6 4-plex RT-qPCR assay. Differences in the performance of the previously developed 3-plex and the newly developed 4-plex RT-qPCR assays were evaluated using the McNemar's test on JMP16 Pro. Statistical significance was set at p-value<0.05.

Group A rotaviruses continue to be a significant cause of diarrhea in children and animal species, including horses [1-6, 40, 41]. Based on the antigenic differences between ERVA genotypes, their spatial and temporal distribution and their impact on vaccine efficacy, molecular surveillance and genotypification of circulating strains is critical to inform on the need for updated vaccines for control and prevention. Most recently, outbreaks of diarrhea in foals associated with ERVB have been detected in Central Kentucky [32]. However, this rotavirus group has been only reported in one out of 37 fecal samples in a single study from Germany [42] and little is known about its distribution, prevalence and pathogenicity compared to ERVA. This new occurrence highlights the emerging potential of this virus and, consequently, diagnostic and epidemiology tools are imperative to understand its biology, epidemiology, virulence, evolution, and ability to generate reassortants. Recently, singleplex TaqMan RT-qPCR assays for ERVB have been described [32] but these have not been thoroughly evaluated or incorporated into existing assays for equine rotavirus diagnostics.

A one-step multiplex (3-plex) TaqMan real-time RT-PCR for the rapid detection and G-typing of the most prevalent genotypes of ERVA (G3 and G14) in fecal specimens. This assay has high sensitivity and specificity, and significantly improved molecular diagnostics for ERVA, reducing turnaround times, labor and laboratory expenses associated with previously used conventional methods for ERVA genotyping, i.e. RT-PCR and Sanger sequencing. With the identification of ERVB in the US, the previously developed ERVA 3-plex assay now incorporates an ERVB-specific target (VP6 or NSP5) to generate, in a single reaction using the TaqMan® chemistry, a 4-plex assay for the simultaneous differentiation between ERVA and ERVB, and the genotypification of the ERVA strains. The present disclosure also encompasses the determination of advantageous the primer and probe concentrations and that of other PCR components such as PCR buffer constituents, dNTPs, and enzyme concentrations in multiplex PCR assay conditions to optimize the specificity, sensitivity, and efficiency of the multiplex assays. This assay rapidly adapts to the needs of the equine industry.

The newly developed assays (ERVA/ERVB VP6 4-plex and ERVA/ERVB NSP5 4-plex) showed a 10-fold higher detection rate limit for the ERVA-specific targets compared to the previously developed ERVA 3-plex assay. This difference could be associated with the modified probe design with optimized dyes and incorporation of an MGB in the design of the G3 VP7-specific prober. While the two assays developed and evaluated here (ERVA/ERVB VP6 and ERVA/ERVB NSP5) had comparable analytical performance, ERVA/ERVB VP6 was selected for clinical performance evaluation over the ERVA/ERVB NSP5 based on its overall higher efficiency among all the targets included in the assay.

The overall sensitivity of the assay for all targets based on its clinical performance was 94% with a specificity of 100%. The sensitivity of the pan-RVA (NSP3) assay was 91.8%, slightly but significantly lower when compared to the previously developed 3-plex assay. This difference could be associated with target competition and exhaustion of reagents during the reaction, which would impact the assay's sensitivity [43]. Such event is suspected of having caused a slightly reduced sensitivity of the pan-RVA (NSP3) component of the assay; however, the sensitivity is still >90%. While the NSP3 target showed this lower sensitivity in the ERVA/ERVB VP6 4-plex assay compared to the 3-plex assay previously developed, the G3 and G14 VP7 targets had comparable sensitivities in the 4-plex and original 3-plex assays.

It is important to note that the three G3 ERVA-positive samples that yielded negative results were the same samples that failed to be genotyped on our previously developed ERVA 3-plex assay, which may be a consequence of low target nucleic acid in these fecal specimens being beyond the limit of detection.

Among the n=7 misidentified samples, (a) n=2 had Ct values of 38 and 39, respectively, with undetermined genotyping results; (b) n=4 yielded an undetermined result but positive detection by the G3/G14 genotyping primer-probe set; and (c) n=1 yielded undetermined results for all targets. The latter specimen has been further tested using a spike-in internal control, which yielded a Ct of ˜27, thus indicating that PCR inhibitors are unlikely to be the source of the failed amplification. Therefore, for the scenario presented under (a) and (c), compromised target integrity is a likely possibility that could have accounted for these results. The scenario presented under (b) could be associated with target competition and exhaustion of reagents during the reaction, which could impact the assay's sensitivity (Bialasiewicz, Whiley, Nissen, & Sloots, 2007); these would still be considered positive following amplification of either G3 or G14. While these discordant samples have caused a slightly reduced sensitivity of the pan-RVA (NSP3) component of the assay, the sensitivity is still >90%. Although the NSP3 target showed this lower sensitivity in the ERVA/ERVB VP6 4-plex assay compared to the 3-plex assay previously developed, the G3 and G14 VP7 targets had comparable sensitivity. Reduced sensitivity of multiplex RT-qPCR or qPCR assays compared to singleplex counterparts is not unusual and has been previously reported (Banerjee et al., 2022; Das et al., 2022; Das, Xu, & Jia, 2019; Elnifro, Ashshi, Cooper, & Klapper, 2000; Furer, Fraefel, & Lechmann, 2022; Gunson, Bennett, Maclean, & Carman, 2008; Pabbaraju, Wong, Ma, Zelyas, & Tipples, 2021; Parker et al., 2018; Vandenbussche et al., 2008). Based on previous studies, the reduction in sensitivity compared to singleplex assays is typically slight could be due to differential amplification of one target over others (based on the amplification efficiencies), target abundance, reagent competition, and non-specific interactions between primer sets, or a combination of these (Das et al., 2022; Das et al., 2019; Elnifro et al., 2000; Gunson et al., 2008). In our previous study, we demonstrated that, in case of low target concentration and high Ct values on the NSP3 assay with no amplification of either genotyping target G3 or G14, genotyping performance can be improved in those cases by performing them under singleplex. This only occurred in a small subset of samples analyzed (3 out of 177; 1.75%) (Carossino et al., 2019). It is important to note that, in the current study, the three G3 ERVA-positive samples that yielded negative results were the same samples that failed to be genotyped on our previously developed ERVA 3-plex assay (Carossino et al., 2019). Thus, low target nucleic acid in these fecal specimens beyond the limit of detection is suspected. Despite this, and with only one out of 193 samples included in the current study (0.52%) in which none of the assays' targets amplified, the 4-plex assay developed here clearly offers a robust, fast, streamlined, and superior tool for surveillance and diagnosis of equine rotaviruses compared to other available tools such as conventional RT-PCR coupled with sequencing, antigen-based enzyme-linked immunosorbent assays (ELISA), or TEM.

Even though a small number of positive samples for ERVB (n=15) could be included in this study, the ERVA/ERVB VP6 4-plex assay was able to correctly detect ERVB in all except one fecal sample (RVB/Horse-wt/USA/KY1-6/2021). VP6 sequencing demonstrated that two of the samples including the one mentioned above showed roughly 4% nucleotide divergence from the reference strain and other ERVB-positive samples sequenced in this study, with a total of four nucleotide substitutions spanning the ERVB VP6 probe and reverse primer sequences used (n=3 and n=1, respectively). However, these differences are unlikely to be the source of the negative result as one of the samples was readily detected by the ERVA/ERVB VP6 4-plex assay. We have tested the extracted RNA with a spike-in internal control for evaluation of PCR inhibitors in this specific fecal sample as a potential cause for the failed amplification and false negative result, however, the internal spike-in control amplified successfully (Ct˜27). Consequently, PCR inhibitors are unlikely to be the source of this failed amplification and low target abundance could have been its source instead. One aspect of the disclosure encompasses embodiments of a panel of oligonucleotides for use in a multiplex reverse transcriptase-polymerase chain reaction (RT-PCR) assay for the identification of rotavirus A and B and genotypes thereof, the panel of nucleotides comprising: a rotavirus A-specific forward PCR primer, a rotavirus A-specific reverse PCR primer, and a labeled oligonucleotide probe, wherein the forward PCR primer, reverse PCR primer, and the labeled oligonucleotide probe are each complementary to a region of a nucleic acid, or the complement thereof, encoding the non-structural protein 3 (NSP3) of an equine rotavirus A; a rotavirus A VP7 (subtype G3 genotype)-specific forward PCR primer, a rotavirus A VP7 (subtype G3 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; a rotavirus A VP7 (subtype G14 genotype)-specific forward PCR primer, a rotavirus A VP7 (subtype G14 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; and either (i) a rotavirus B VP6-specific forward PCR primer, a rotavirus B VP6-specific reverse PCR primer, and a labeled oligonucleotide, wherein the forward PCR primer, reverse PCR primer, and the labeled oligonucleotide probe are each complementary to a nucleic acid, or the complement thereof, encoding the structural protein VP6, or a fragment thereof, of an equine rotavirus B, or (ii) a rotavirus B non-structural protein 5 (NSP5)-specific forward PCR primer, a rotavirus B an NSP5-specific reverse PCR primer, and a labeled oligonucleotide probe, wherein the forward PCR primer, reverse PCR primer, and the labeled oligonucleotide probe are each complementary to a nucleic acid, or the complement thereof, encoding the non-structural protein NSP5, or a fragment thereof, of an equine rotavirus B.

In some embodiments of this aspect of the disclosure, the primers and the labeled oligonucleotide probes can be selected from the group consisting of: NVP3-FDeg, ACCATCTWCACRTRACCCTC (SEQ ID NO:29); NVP3-R1, GGTCACATAACGCCCCTATA (SEQ ID NO:30); NVP3-Probe, JUN-ATGAGCACAATAGTTAAAAGCTAACACTGTCAA-QSY (SEQ ID NO:31); RVA-G3-756F, GATGTTACCACGACCACTTGTA (SEQ ID NO:32); RVA-G3-872R, AGTTGGATCGGCCGTTATG (SEQ ID NO:33); RVA-G3-779P, FAM-TGGGACCACGAGAGAATGTAGCTGT-MGB (SEQ ID NO:34); RVA-G14-ARG869F, ATCCGACTACGGCTCCA (SEQ ID NO:35); RVA-G14-ARG1011R, TGCAGCAGAATTTAATGATCGC (SEQ ID NO:36); RVA-G14-ARG886P, VIC-CAGATTGGACGAATGATGCGTATAAATTGG-MGB (SEQ ID NO:37); ERVB-VP6-F, CATCCAGAGTGAATGGGAAGAC (SEQ ID NO:38); ERVB-VP6-R, TTCTAACGGCCAGCGAAATTA (SEQ ID NO:39); ERVB-VP6-P, LIZ-CCCTTACACGATACACGCACCGA-QSY (SEQ ID NO:40); ERVB-NSP5-F, GCCTTCTGATTCTACGTCAACTA (SEQ ID NO:41); ERVB-NSP5-R, CTTGTTGTACGCTTCTTCGTATTC (SEQ ID NO:42); and ERVB-NSP5-P, LIZ-AACATCAAGTCGTAGCGACGCAGT-QSY (SEQ ID NO:43), wherein each of the labeled oligonucleotide probes has a detectable label conjugated at each of the 5′ and the 3′ termini of the oligonucleotide.

In some embodiments of this aspect of the disclosure, the detectable label conjugated at each of the 5′ and the 3′ termini of the oligonucleotide can be selected from the group consisting of FAM, 6-carboxyfluorescein; JUN, JUN® dye; LIZ, LIZ® dye; MGB, minor groove binder; QSY, QSY® quencher; and VIC, VIC® dye.

Another aspect of the disclosure encompasses embodiments of an RT-PCR multiplex method for determining whether an equine is infected with an equine rotavirus, the method comprising the steps of: (a) obtaining a fecal sample from the equine; (b) extracting RNA from the fecal sample; (c) assaying the RNA by an RT-PCR multiplex assay performed with a panel of nucleotides comprising: a rotavirus A-specific forward PCR primer, a rotavirus A-specific reverse PCR primer, and a labeled oligonucleotide probe, wherein the forward PCR primer, reverse PCR primer, and the labeled oligonucleotide probe are each complementary to a region of a nucleic acid, or the complement thereof, encoding the non-structural protein 3 (NSP3) of an equine rotavirus A; a rotavirus A VP7 (subtype G3 genotype)-specific forward PCR primer, a rotavirus A VP7 (subtype G3 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; a rotavirus A VP7 (subtype G14 genotype)-specific forward PCR primer, a rotavirus A VP7 (subtype G14 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; and either (i) a rotavirus B VP6-specific forward PCR primer, a rotavirus B VP6-specific reverse PCR primer, and a labeled oligonucleotide, wherein the forward PCR primer, reverse PCR primer, and the labeled oligonucleotide probe are each complementary to a nucleic acid, or the complement thereof, encoding the non-structural protein VP6, or a fragment thereof, of an equine rotavirus B, or (ii) a rotavirus B non-structural protein 5 (NSP5)-specific forward PCR primer, a rotavirus B an NSP5-specific reverse PCR primer, and a labeled oligonucleotide probe, wherein the forward PCR primer, reverse PCR primer, and the labeled oligonucleotide probe are each complementary to a nucleic acid, or the complement thereof, encoding the non-structural protein NSP5, or a fragment thereof, of an equine rotavirus B, wherein detection of the rotavirus A or B indicates an infection of the equine with the rotavirus A or B and whether, if present, the rotavirus A is of the genotype VP7 subtype G3 or G14.

In some embodiments of this aspect of the disclosure, the primers and the labeled oligonucleotide probes can be selected from the group consisting of: NVP3-FDeg, ACCATCTWCACRTRACCCTC; NVP3-R1, GGTCACATAACGCCCCTATA; NVP3-Probe, JUN-ATGAGCACAATAGTTAAAAGCTAACACTGTCAA-QSY; RVA-G3-756F, GATGTTACCACGACCACTTGTA; RVA-G3-872R, AGTTGGATCGGCCGTTATG; RVA-G3-779P, FAM-TGGGACCACGAGAGAATGTAGCTGT-MGB; RVA-G14-ARG869F, ATCCGACTACGGCTCCA; RVA-G14-ARG1011R, TGCAGCAGAATTTAATGATCGC; RVA-G14-ARG886P, VIC-CAGATTGGACGAATGATGCGTATAAATTGG-MGB; ERVB-VP6-F, CATCCAGAGTGAATGGGAAGAC; ERVB-VP6-R, TTCTAACGGCCAGCGAAATTA; ERVB-VP6-P, LIZ-CCCTTACACGATACACGCACCGA-QSY; ERVB-NSP5-F, GCCTTCTGATTCTACGTCAACTA; ERVB-NSP5-R, CTTGTTGTACGCTTCTTCGTATTC; and ERVB-NSP5-P, LIZ-AACATCAAGTCGTAGCGACGCAGT-QSY, wherein each of the labeled oligonucleotide probes has a detectable label conjugated at each of the 5′ and the 3′ termini of the oligonucleotide and wherein detection of the rotavirus A or B indicates an infection of the equine with the rotavirus A or B and whether, if present, the rotavirus A is of the genotype VP7 subtype G3 or G14.

Sequences

RVB/Horse-wt/USA/KY1-1/2021:
AGATTGCAGAAAAGAGTATTATCACTAGCTCCAAACACAAACTTGAACACTGCAGGT
CAGTCAATTCTCAATGATTATAATGCTATAGCATCCAGAGTGAATGGGAAGACTTAT
GCTCTTTTGGACCAAACAGCAATATTATCCCCTTACACGATACACGCACCGATAATT
TCGCTGGCCGTTAGAATATCTACTGATGATTATGATGACATGAGAAATGGAGTTGAG
TCTATACTAGATTGTTTGGCTGCGGCGATTCGCACTGAAGGCTCGAGACCGGTTAG
AGTGATTGAACGTAGAGTTATTGAACCAGTGGTAAAGCAGCTGGTCGAAGATCTGA
AGTTAAAAAGTCTGATTTCTGAAATCTCAATTGCCAATTTCGCTGCTACTGATACCG
CGCTTATCCAACCAGAAGTAGTAGAAACTGAAAATCCATTGATAGTTGGTATCATAG
AACAGGTGGTTGTAAGACAACCAGCCAGTCTAAATGGTGGCAATATTAGAGCAGCG
ATTGGCAGATGGTCAGGTAATAAAGGCTCAGTCACATGTGTCTCAGGCATGGAAGC
AGAACATATGTTCTTCGTGGAACTAAAAGCTAGGACGTGTGGTGTACTGAACGTCG
TTTATCTGCCAGCCCCAGGAGTTATAATGGTGCCTATGCCGCAAGGACGCAACAGA
GAAAGTGTTATACTTGACGTATCCGCAGAGATGACAGCAGATGATTTTATAATCGAT
TTCTTTGATGATAACAACATTGTCCATACGGAAAGAGGAGTTGGCCTATTTTCATTT
CCAATGTGTACCAGAATTAGATTTAGAGTTACACCATGGACACAACAAAAATCTCAG
AATGGACTTGACACTCCATCATTGGCTACGTGGGCGAACGGTACGTCTCCGAGGC
AGCCAGCGGTGTCTTTCATGTTTGAATTAAGAAGAACCTTCACTGAAAACGATTATA
AATTCGTTTCACGATGTACCTCGAAAGTTCAATATATATTGGATACCAACTTCCCAG
AGACATCATTTATTAACAGGCCTCAAATAGAATGGAACGTACAAGAGATGATTACTT
CTGACACAGACACAGTATGGTCACGTAAAATCGCAATGCTAGTCGCAGCATTTGCT
GCTAAGATCTGATTCTCC (SEQ ID NO: 1).
RVB/Horse-wt/USA/KY1-2/2021:
CGTCAGATTGCAGAAAAGAGTATTATCACTAGCTCCAAACACAAACTTGAACACTGC
AGGTCAGTCAATTCTCAATGATTATAATGCTATAGCATCCAGAGTGAATGGGAAGAC
TTATGCTCTTTTGGACCAAACAGCAATATTATCCCCTTACACGATACACGCACCGAT
AATTTCGCTGGCCGTTAGAATATCTACTGATGATTATGATGACATGAGAAATGGAGT
TGAGTCTATACTAGATTGTTTGGCTGCGGCGATTCGCACTGAAGGCTCGAGACCGG
TTAGAGTGATTGAACGTAGAGTTATTGAACCAGTGGTAAAGCAGCTGGTCGAAGAT
CTGAAGTTAAAAAGTCTGATTTCTGAAATCTCAATTGCCAATTTCGCTGCTACTGATA
CCGCGCTTATCCAACCAGAAGTAGTAGAAACTGAAAATCCATTGATAGTTGGTATCA
TAGAACAGGTGGTTGTAAGACAACCAGCCAGTCTAAATGGTGGCAATATTAGAGCA
GCGATTGGCAGATGGTCAGGTAATAAAGGCTCAGTCACATGTGTCTCAGGCATGGA
AGCAGAACATATGTTCTTCGTGGAACTAAAAGCTAGGACGTGTGGTGTACTGAACG
TCGTTTATCTGCCAGCCCCAGGAGTTATAATGGTGCCTATGCCGCAAGGACGCAAC
AGAGAAAGTGTTATACTTGACGTATCCGCAGAGATGACAGCAGATGATTTTATAATC
GATTTCTTTGATGATAACAACATTGTCCATACGGAAAGAGGAGTTGGCCTATTTTCA
TTTCCAATGTGTACCAGAATTAGATTTAGAGTTACACCATGGACACAACAAAAATCT
CAGAATGGACTTGACACTCCATCATTGGCTACGTGGGCGAACGGTACGTCTCCGA
GGCAGCCAGCGGTGTCTTTCATGTTTGAATTAAGAAGAACCTTCACTGAAAACGATT
ATAAATTCGTTTCACGATGTACCTCGAAAGTTCAATATATATTGGATACCAACTTCCC
AGAGACATCATTTATTAACAGGCCTCAAATAGAATGGAACGTACAAGAGATGATTAC
TTCTGACACAGACACAGTATGGTCACGTAAAATCGCAATGCTAGTCGCAGCATTTG
CTGCTAAGATCTGATTCT (SEQ ID NO: 2).
RVB/Horse-wt/USA/KY1-3/2021:
CAGATTGCAGAAAAGAGTATTATCACTAGCTCCAAACACAAACTTGAACACTGCAGG
TCAGTCAATTCTCAATGATTATAATGCTATAGCATCCAGAGTGAATGGGAAGACTTA
TGCTCTTTTGGACCAAACAGCAATATTATCCCCTTACACGATACACGCACCGATAAT
TTCGCTGGCCGTTAGAATATCTACTGATGATTATGATGACATGAGAAATGGAGTTGA
GTCTATACTAGATTGTTTGGCTGCGGCGATTCGCACTGAAGGCTCGAGACCGGTTA
GAGTGATTGAACGTAGAGTTATTGAACCAGTGGTAAAGCAGCTGGTCGAAGATCTG
AAGTTAAAAAGTCTGATTTCTGAAATCTCAATTGCCAATTTCGCTGCTACTGATACC
GCGCTTATCCAACCAGAAGTAGTAGAAACTGAAAATCCATTGATAGTTGGTATCATA
GAACAGGTGGTTGTAAGACAACCAGCCAGTCTAAATGGTGGCAATATTAGAGCAGC
GATTGGCAGATGGTCAGGTAATAAAGGCTCAGTCACATGTGTCTCAGGCATGGAAG
CAGAACATATGTTCTTCGTGGAACTAAAAGCTAGGACGTGTGGTGTACTGAACGTC
GTTTATCTGCCAGCCCCAGGAGTTATAATGGTGCCTATGCCGCAAGGACGCAACAG
AGAAAGTGTTATACTTGACGTATCCGCAGAGATGACAGCAGATGATTTTATAATCGA
TTTCTTTGATGATAACAACATTGTCCATACGGAAAGAGGAGTTGGCCTATTTTCATTT
CCAATGTGTACCAGAATTAGATTTAGAGTTACACCATGGACACAACAAAAATCTCAG
AATGGACTTGACACTCCATCATTGGCTACGTGGGCGAACGGTACGTCTCCGAGGC
AGCCAGCGGTGTCTTTCATGTTTGAATTAAGAAGAACCTTCACTGAAAACGATTATA
AATTCGTTTCACGATGTACCTCGAAAGTTCAATATATATTGGATACCAACTTCCCAG
AGACATCATTTATTAACAGGCCTCAAATAGAATGGAACGTACAAGAGATGATTACTT
CTGACACAGACACAGTATGGTCACGTAAAATCGCAATGCTAGTCGCAGCATTTGCT
GCTAAGATCTGATTCTC (SEQ ID NO: 3).
RVB/Horse-wt/USA/KY1-4/2021:
CGTCAGATTGCAGAAAAGAGTATTATCACTAGCTCCAAACACAAACTTGAACACTGC
AGGTCAGTCAATTCTCAATGATTATAATGCTATAGCATCCAGAGTGAATGGGAAGAC
TTATGCTCTTTTGGACCAAACAGCAATATTATCCCCTTACACGATACACGCACCGAT
AATTTCGCTGGCCGTTAGAATATCTACTGATGATTATGATGACATGAGAAATGGAGT
TGAGTCTATACTAGATTGTTTGGCTGCGGCGATTCGCACTGAAGGCTCGAGACCGG
TTAGAGTGATTGAACGTAGAGTTATTGAACCAGTGGTAAAGCAGCTGGTCGAAGAT
CTGAAGTTAAAAAGTCTGATTTCTGAAATCTCAATTGCCAATTTCGCTGCTACTGATA
CCGCGCTTATCCAACCAGAAGTAGTAGAAACTGAAAATCCATTGATAGTTGGTATCA
TAGAACAGGTGGTTGTAAGACAACCAGCCAGTCTAAATGGTGGCAATATTAGAGCA
GCGATTGGCAGATGGTCAGGTAATAAAGGCTCAGTCACATGTGTCTCAGGCATGGA
AGCAGAACATATGTTCTTCGTGGAACTAAAAGCTAGGACGTGTGGTGTACTGAACG
TCGTTTATCTGCCAGCCCCAGGAGTTATAATGGTGCCTATGCCGCAAGGACGCAAC
AGAGAAAGTGTTATACTTGACGTATCCGCAGAGATGACAGCAGATGATTTTATAATC
GATTTCTTTGATGATAACAACATTGTCCATACGGAAAGAGGAGTTGGCCTATTTTCA
TTTCCAATGTGTACCAGAATTAGATTTAGAGTTACACCATGGACACAACAAAAATCT
CAGAATGGACTTGACACTCCATCATTGGCTACGTGGGCGAACGGTACGTCTCCGA
GGCAGCCAGCGGTGTCTTTCATGTTTGAATTAAGAAGAACCTTCACTGAAAACGATT
ATAAATTCGTTTCACGATGTACCTCGAAAGTTCAATATATATTGGATACCAACTTCCC
AGAGACATCATTTATTAACAGGCCTCAAATAGAATGGAACGTACAAGAGATGATTAC
TTCTGACACAGACACAGTATGGTCACGTAAAATCGCAATGCTAGTCGCAGCATTTG
CTGCTAAGATCTGATTCTC (SEQ ID NO: 4).
RVB/Horse-wt/USA/KY1-5/2021:
CAGATTGCAGAAAAGAGTATTATCACTAGCTCCAAACACAAACTTGAACACTGCAGG
TCAGTCAATTCTCAATGATTATAATGCTATAGCATCCAGAGTGAATGGGAAGACTTA
TGCTCTTTTGGACCAAACAGCAATATTATCCCCTTACACGATACACGCACCGATAAT
TTCGCTGGCCGTTAGAATATCTACTGATGATTATGATGACATGAGAAATGGAGTTGA
GTCTATACTAGATTGTTTGGCTGCGGCGATTCGCACTGAAGGCTCGAGACCGGTTA
GAGTGATTGAACGTAGAGTTATTGAACCAGTGGTAAAGCAGCTGGTCGAAGATCTG
AAGTTAAAAAGTCTGATTTCTGAAATCTCAATTGCCAATTTCGCTGCTACTGATACC
GCGCTTATCCAACCAGAAGTAGTAGAAACTGAAAATCCATTGATAGTTGGTATCATA
GAACAGGTGGTTGTAAGACAACCAGCCAGTCTAAATGGTGGCAATATTAGAGCAGC
GATTGGCAGATGGTCAGGTAATAAAGGCTCAGTCACATGTGTCTCAGGCATGGAAG
CAGAACATATGTTCTTCGTGGAACTAAAAGCTAGGACGTGTGGTGTACTGAACGTC
GTTTATCTGCCAGCCCCGGGAGTTATAATGGTGCCTATGCCGCAAGGACGCAACA
GAGAAAGTGTTATACTTGACGTATCCGCAGAGATGACAGCAGATGATTTTATAATCG
ATTTCTTTGATGATAACAACATTGTCCATACGGAAAGAGGAGTTGGCCTATTTTCATT
TCCAATGTGTACCAGAATTAGATTTAGAGTTACACCATGGACACAACAAAAATCTCA
GAATGGACTTGACACTCCATCATTGGCTACGTGGGCGAACGGTACGTCTCCGAGG
CAGCCAGCGGTGTCTTTCATGTTTGAATTAAGAAGAACCTTCACTGAAAACGATTAT
AAATTCGTTTCACGATGTACCTCGAAAGTTCAATACATATTGGATACCAACTTCCCA
GAGACATCATTTATTAACAGGCCTCAAATAGAATGGAACGTACAAGAGATGATTACT
TCTGACACAGACACAGTATGGTCACGTAAAATCGCAATGCTAGTCGCAGCATTTGC
TGCTAAGATCTGATT (SEQ ID NO: 5).
RVB/Horse-wt/USA/KY1-6/2021:
GCGTCAGATTGCAGAAAAGAGTATTATCACTAGCTCCAAACACAAACTTGAACACTG
CAGGTCAGTCAATTCTCAATGATTACAATGCCATAGCATCCAGAGTGAATGGGAAG
ACTTATGCTCTTTTGGACCAAACGGCAATATTATCCCCTTACACTATACATGCACCA
ATAATTTCACTGGCCGTTAGAATATCTACTGATGATTATGATGACATGAGAAATGGA
GTTGAGTCTATACTAGACTGTTTGGCTGCGGCAATTCGTACTGAAGGCTCGAGACC
GGTTAGAGTGATTGAACGTAGGGTTATTGAACCAGTGGTAAAACAGCTGGTCGAAG
ATCTGAAGTTGAAAAGTCTAGTTTCTGAAATATCAATTGCCAATTTCGCTGCTGCCG
ATACTGCGCTTATTCAACCAGAGATAGTAGAAACTGAAAATCCATTAATAGTTGGTA
TCATAGAACAGGTGGTTGTAAGACAACCAGCCAGTCTAAATGGTGGTAATATTAGA
GCAGCGATTGGCAGGTGGTCAGGTAATAAAGGCTCAGTCACATGTGTCTCAGGCAT
GGAAGCAGAACATATGTTTTTTGTGGAACTAAAAGCTAGAACGTGTGGTGTGCTGA
ACGTCGTTTATCTGCCAGCTCCAGGAGTTATAATGGTGCCTATGCCGCAAGGACGC
AACAGAGAAAGTGTTATACTTGACGTATCCGCAGAGATGACAGCAGATGATTTTATA
ATTGATTTCTTTGATGACAACAACATTGTTCATACGGAAAGAGGAGTTGGCCTATTT
TCATTTCCAATGTGTACCAGAATTAGGTTTAGAGTTACACCATGGACACAGCAAAAA
TCTCAGAATGGACTTGACACTCCATCATTGGCTACGTGGGCGAACGGCACATCTCC
GAGACAGCCAGCGGTGTCTTTCATGTTTGAATTAAGAAGAACCTTCACTGAAAATGA
TTATAAATTCGTTTCAAGATGTACCTCAAAAGTTCAATACATATTGGATACCAACTTC
CCAGAGACATCTTTTATTAACAGGCCTCAAATAGAATGGAACGTACAAGAGATGATT
ACTTCTGACACTGATACAGTATGGTCACGTAAAATCGCAATGCTAGTCGCAGCATTT
GCTGCTAAGATCTGATTC (SEQ ID NO: 6).
RVB/Horse-wt/USA/KY1-7/2021:
CAGATTGCAGAAAAGAGTATTATCACTAGCTCCAAACACAAACTTGAACACTGCAGG
TCAGTCAATTCTCAATGATTATAATGCTATAGCATCCAGAGTGAATGGGAAGACTTA
TGCTCTTTTGGACCAAACAGCAATATTATCCCCTTACACGATACACGCACCGATAAT
TTCGCTGGCCGTTAGAATATCTACTGATGATTATGATGACATGAGAAATGGAGTTGA
GTCTATACTAGATTGTTTGGCTGCGGCGATTCGCACTGAAGGCTCGAGACCGGTTA
GAGTGATTGAACGTAGAGTTATTGAACCAGTGGTAAAGCAGCTGGTCGAAGATCTG
AAGTTAAAAAGTCTGATTTCTGAAATCTCAATTGCCAATTTCGCTGCTACTGATACC
GCGCTTATCCAACCAGAAGTAGTAGAAACTGAAAATCCATTGATAGTTGGTATCATA
GAACAGGTGGTTGTAAGACAACCAGCCAGTCTAAATGGTGGCAATATTAGAGCAGC
GATTGGCAGATGGTCAGGTAATAAAGGCTCAGTCACATGTGTCTCAGGCATGGAAG
CAGAACATATGTTCTTCGTGGAACTAAAAGCTAGGACGTGTGGTGTACTGAACGTC
GTTTATCTGCCAGCCCCGGGAGTTATAATGGTGCCTATGCCGCAAGGACGCAACA
GAGAAAGTGTTATACTTGACGTATCCGCAGAGATGACAGCAGATGATTTTATAATCG
ATTTCTTTGATGATAACAACATTGTCCATACGGAAAGAGGAGTTGGCCTATTTTCATT
TCCAATGTGTACCAGAATTAGATTTAGAGTTACACCATGGACACAACAAAAATCTCA
GAATGGACTTGACACTCCATCATTGGCTACGTGGGCGAACGGTACGTCTCCGAGG
CAGCCAGCGGTGTCTTTCATGTTTGAATTAAGAAGAACCTTCACTGAAAACGATTAT
AAATTCGTTTCACGATGTACCTCGAAAGTTCAATACATATTGGATACCAACTTCCCA
GAGACATCATTTATTAACAGGCCTCAAATAGAATGGAACGTACAAGAGATGATTACT
TCTGACACAGACACAGTATGGTCACGTAAAATCGCAATGCTAGTCGCAGCATTTGC
TGCTAAGATCTGATT (SEQ ID NO: 7).
RVB/Horse-wt/USA/KY1-9/2021:
GCGTCAGATTGCAGAAAAGAGTATTATCACTAGCTCCAAACACAAACTTGAACACTG
CAGGTCAGTCAATTCTCAATGATTATAATGCTATAGCATCCAGAGTGAATGGGAAGA
CTTATGCTCTTTTGGACCAAACAGCAATATTATCCCCTTACACGATACACGCACCGA
TAATTTCGCTGGCCGTTAGAATATCTACTGATGATTATGATGACATGAGAAATGGAG
TTGAGTCTATACTAGATTGTTTGGCTGCGGCGATTCGCACTGAAGGCTCGAGACCG
GTTAGAGTGATTGAACGTAGAGTTATTGAACCAGTGGTAAAGCAGCTGGTCGAAGA
TCTGAAGTTAAAAAGTCTGATTTCTGAAATCTCAATTGCCAATTTCGCTGCTACTGAT
ACCGCGCTTATCCAACCAGAAGTAGTAGAAACTGAAAATCCATTGATAGTTGGTATC
ATAGAACAGGTGGTTGTAAGACAACCAGCCAGTCTAAATGGTGGCAATATTAGAGC
AGCGATTGGCAGATGGTCAGGTAATAAAGGCTCAGTCACATGTGTCTCAGGCATGG
AAGCAGAACATATGTTCTTCGTGGAACTAAAAGCTAGGACGTGTGGTGTACTGAAC
GTCGTTTATCTGCCAGCCCCAGGAGTTATAATGGTGCCTATGCCGCAAGGACGCAA
CAGAGAAAGTGTTATACTTGACGTATCCGCAGAGATGACAGCAGATGATTTTATAAT
CGATTTCTTTGATGATAACAACATTGTCCATACGGAAAGAGGAGTTGGCCTATTTTC
ATTTCCAATGTGTACCAGAATTAGATTTAGAGTTACACCATGGACACAACAAAAATC
TCAGAATGGACTTGACACTCCATCATTGGCTACGTGGGCGAACGGTACGTCTCCGA
GGCAGCCAGCGGTGTCTTTCATGTTTGAATTAAGAAGAACCTTCACTGAAAACGATT
ATAAATTCGTTTCACGATGTACCTCGAAAGTTCAATATATATTGGATACCAACTTCCC
AGAGACATCATTTATTAACAGGCCTCAAATAGAATGGAACGTACAAGAGATGATTAC
TTCTGACACAGACACAGTATGGTCACGTAAAATCGCAATGCTAGTCGCAGCATTTG
CTGCTAAGATCTGATTCTCC (SEQ ID NO: 8).
RVB/Horse-wt/USA/KY1-10/2021:
CAGATTGCAGAAAAGAGTATTATCACTAGCTCCAAACACAAACTTGAACACTGCAGG
TCAGTCAATTCTCAATGATTATAATGCTATAGCATCCAGAGTGAATGGGAAGACTTA
TGCTCTTTTGGACCAAACAGCAATATTATCCCCTTACACGATACACGCACCGATAAT
TTCGCTGGCCGTTAGAATATCTACTGATGATTATGATGACATGAGAAATGGAGTTGA
GTCTATACTAGATTGTTTGGCTGCGGCGATTCGCACTGAAGGCTCGAGACCGGTTA
GAGTGATTGAACGTAGAGTTATTGAACCAGTGGTAAAGCAGCTGGTCGAAGATCTG
AAGTTAAAAAGTCTGATTTCTGAAATCTCAATTGCCAATTTCGCTGCTACTGATACC
GCGCTTATCCAACCAGAAGTAGTAGAAACTGAAAATCCATTGATAGTTGGTATCATA
GAACAGGTGGTTGTAAGACAACCAGCCAGTCTAAATGGTGGCAATATTAGAGCAGC
GATTGGCAGATGGTCAGGTAATAAAGGCTCAGTCACATGTGTCTCAGGCATGGAAG
CAGAACATATGTTCTTCGTGGAACTAAAAGCTAGGACGTGTGGTGTACTGAACGTC
GTTTATCTGCCAGCCCCGGGAGTTATAATGGTGCCTATGCCGCAAGGACGCAACA
GAGAAAGTGTTATACTTGACGTATCCGCAGAGATGACAGCAGATGATTTTATAATCG
ATTTCTTTGATGATAACAACATTGTCCATACGGAAAGAGGAGTTGGCCTATTTTCATT
TCCAATGTGTACCAGAATTAGATTTAGAGTTACACCATGGACACAACAAAAATCTCA
GAATGGACTTGACACTCCATCATTGGCTACGTGGGCGAACGGTACGTCTCCGAGG
CAGCCAGCGGTGTCTTTCATGTTTGAATTAAGAAGAACCTTCACTGAAAACGATTAT
AAATTCGTTTCACGATGTACCTCGAAAGTTCAATACATATTGGATACCAACTTCCCA
GAGACATCATTTATTAACAGGCCTCAAATAGAATGGAACGTACAAGAGATGATTACT
TCTGACACAGACACAGTATGGTCACGTAAAATCGCAATGCTAGTCGCAGCATTTGC
TGCTAAGATCTGATT (SEQ ID NO: 9).
RVB/Horse-wt/USA/KY1-11/2021:
CGTCAGATTGCAGAAAAGAGTATTATCACTAGCTCCAAACACAAACTTGAACACTGC
AGGTCAGTCAATTCTCAATGATTATAATGCTATAGCATCCAGAGTGAATGGGAAGAC
TTATGCTCTTTTGGACCAAACAGCAATATTATCCCCTTACACGATACACGCACCGAT
AATTTCGCTGGCCGTTAGAATATCTACTGATGATTATGATGACATGAGAAATGGAGT
TGAGTCTATACTAGATTGTTTGGCTGCGGCGATTCGCACTGAAGGCTCGAGACCGG
TTAGAGTGATTGAACGTAGAGTTATTGAACCAGTGGTAAAGCAGCTGGTCGAAGAT
CTGAAGTTAAAAAGTCTGATTTCTGAAATCTCAATTGCCAATTTCGCTGCTACTGATA
CCGCGCTTATCCAACCAGAAGTAGTAGAAACTGAAAATCCATTGATAGTTGGTATCA
TAGAACAGGTGGTTGTAAGACAACCAGCCAGTCTAAATGGTGGCAATATTAGAGCA
GCGATTGGCAGATGGTCAGGTAATAAAGGCTCAGTCACATGTGTCTCAGGCATGGA
AGCAGAACATATGTTCTTCGTGGAACTAAAAGCTAGGACGTGTGGTGTACTGAACG
TCGTTTATCTGCCAGCCCCAGGAGTTATAATGGTGCCTATGCCGCAAGGACGCAAC
AGAGAAAGTGTTATACTTGACGTATCCGCAGAGATGACAGCAGATGATTTTATAATC
GATTTCTTTGATGATAACAACATTGTCCATACGGAAAGAGGAGTTGGCCTATTTTCA
TTTCCAATGTGTACCAGAATTAGATTTAGAGTTACACCATGGACACAACAAAAATCT
CAGAATGGACTTGACACTCCATCATTGGCTACGTGGGCGAACGGTACGTCTCCGA
GGCAGCCAGCGGTGTCTTTCATGTTTGAATTAAGAAGAACCTTCACTGAAAACGATT
ATAAATTCGTTTCACGATGTACCTCGAAAGTTCAATATATATTGGATACCAACTTCCC
AGAGACATCATTTATTAACAGGCCTCAAATAGAATGGAACGTACAAGAGATGATTAC
TTCTGACACAGACACAGTATGGTCACGTAAAATCGCAATGCTAGTCGCAGCATTTG
CTGCTAAGATCTGA (SEQ ID NO: 10).
RVB/Horse-wt/USA/KY1-12/2021:
GCGTCAGATTGCAGAAAAGAGTATTATCACTAGCTCCAAACACAAACTTGAACACTG
CAGGTCAGTCAATTCTCAATGATTATAATGCTATAGCATCCAGAGTGAATGGGAAGA
CTTATGCTCTTTTGGACCAAACAGCAATATTATCCCCTTACACGATACACGCACCGA
TAATTTCGCTGGCCGTTAGAATATCTACTGATGATTATGATGACATGAGAAATGGAG
TTGAGTCTATACTAGATTGTTTGGCTGCGGCGATTCGCACTGAAGGCTCGAGACCG
GTTAGAGTGATTGAACGTAGAGTTATTGAACCAGTGGTAAAGCAGCTGGTCGAAGA
TCTGAAGTTAAAAAGTCTGATTTCTGAAATCTCAATTGCCAATTTCGCTGCTACTGAT
ACCGCGCTTATCCAACCAGAAGTAGTAGAAACTGAAAATCCATTGATAGTTGGTATC
ATAGAACAGGTGGTTGTAAGACAACCAGCCAGTCTAAATGGTGGCAATATTAGAGC
AGCGATTGGCAGATGGTCAGGTAATAAAGGCTCAGTCACATGTGTCTCAGGCATGG
AAGCAGAACATATGTTCTTCGTGGAACTAAAAGCTAGGACGTGTGGTGTACTGAAC
GTCGTTTATCTGCCAGCCCCAGGAGTTATAATGGTGCCTATGCCGCAAGGACGCAA
CAGAGAAAGTGTTATACTTGACGTATCCGCAGAGATGACAGCAGATGATTTTATAAT
CGATTTCTTTGATGATAACAACATTGTCCATACGGAAAGAGGAGTTGGCCTATTTTC
ATTTCCAATGTGTACCAGAATTAGATTTAGAGTTACACCATGGACACAACAAAAATC
TCAGAATGGACTTGACACTCCATCATTGGCTACGTGGGCGAACGGTACGTCTCCGA
GGCAGCCAGCGGTGTCTTTCATGTTTGAATTAAGAAGAACCTTCACTGAAAACGATT
ATAAATTCGTTTCACGATGTACCTCGAAAGTTCAATATATATTGGATACCAACTTCCC
AGAGACATCATTTATTAACAGGCCTCAAATAGAATGGAACGTACAAGAGATGATTAC
TTCTGACACAGACACAGTATGGTCACGTAAAATCGCAATGCTAGTCGCAGCATTTG
CTGCTAAGATCTGATTCTCC (SEQ ID NO: 11).
RVB/Horse-wt/USA/KY1-13/2021:
GTGAACGCTTGCGTCAGATTGCAGAAAAGAGTATTATCACTAGCTCCAAACACAAA
CTTGAACACTGCAGGTCAGTCAATTCTCAATGATTACAATGCCATAGCATCCAGAGT
GAATGGGAAGACTTATGCTCTTTTGGACCAAACGGCAATATTATCCCCTTACACTAT
ACATGCACCAATAATTTCACTGGCCGTTAGAATATCTACTGATGATTATGATGACAT
GAGAAATGGAGTTGAGTCTATACTAGACTGTTTGGCTGCGGCAATTCGTACTGAAG
GCTCGAGACCGGTTAGAGTGATTGAGCGTAGGGTTATTGAACCAGTGGTAAAACAG
CTGGTCGAAGATCTGAAGTTGAAAAGTCTAGTTTCTGAAATATCAATTGCCAATTTC
GCTGCTGCCGATACTGCGCTTATTCAACCAGAGATAGTAGAAACTGAAAATCCATTA
ATAGTTGGTATCATAGAACAGGTGGTTGTAAGACAACCAGCCAGTCTAAATGGTGG
TAATATTAGAGCAGCGATTGGCAGGTGGTCAGGAATAAAGGCTCAGTCACATGTGT
CTCAGGCATGGAAGCAGAACATATGTTTTTTGTGGAACTAAAAGCTAGAACGTGTG
GTGTGCTGAACGTCGTTTATCTGCCAGCTCCAGGAGTTATAATGGTGCCTATGCCG
CAAGGACGCAACAGAGAAAGTGTTATACTTGACGTATCCGCAGAGATGACAGCAGA
TGATTTTATAATTGATTTCTTTGATGACAACAACATTGTTCATACGGAAAGAGGAGTT
GGCCTATTTTCATTTCCAATGTGTACCAGAATTAGGTTTAGAGTTACACCATGGACA
CAGCAAAAATCTCAGAATGGACTTGACACTCCATCATTGGCTACGTGGGCGAACGG
CACATCTCCGAGACAGCCAGCGGTGTCTTTCATGTTTGAATTAAGAAGAACCTTCAC
TGAAAATGATTACAAATTCGTTTCAAGATGTACCTCAAAAGTTCAATACATATTGGAT
ACCAACTTCCCAGAGACATCTTTTATTAACAGGCCTCAAATAGAATGGAACGTACAA
GAGATGATTACTTCTGACACTGATACAGTATGGTCACGTAAAATCGCAATGCTAGTC
GCAGCATTTGCTGCTAAGATCTGATTCTC (SEQ ID NO: 12).
RVB/Horse-wt/USA/KY1-14/2021:
AGATTGCAGAAAAGAGTATTATCACTAGCTCCAAACACAAACTTGAACACTGCAGGT
CAGTCAATTCTCAATGATTATAATGCTATAGCATCCAGAGTGAATGGGAAGACTTAT
GCTCTTTTGGACCAAACAGCAATATTATCCCCTTACACGATACACGCACCGATAATT
TCGCTGGCCGTTAGAATATCTACTGATGATTATGATGACATGAGAAATGGAGTTGAG
TCTATACTAGATTGTTTGGCTGCGGCGATTCGCACTGAAGGCTCGAGACCGGTTAG
AGTGATTGAACGTAGAGTTATTGAACCAGTGGTAAAGCAGCTGGTCGAAGATCTGA
AGTTAAAAAGTCTGATTTCTGAAATCTCAATTGCCAATTTCGCTGCTACTGATACCG
CGCTTATCCAACCAGAAGTAGTAGAAACTGAAAATCCATTGATAGTTGGTATCATAG
AACAGGTGGTTGTAAGACAACCAGCCAGTCTAAATGGTGGCAATATTAGAGCAGCG
ATTGGCAGATGGTCAGGTAATAAAGGCTCAGTCACATGTGTCTCAGGCATGGAAGC
AGAACATATGTTCTTCGTGGAACTAAAAGCTAGGACGTGTGGTGTACTGAACGTCG
TTTATCTGCCAGCCCCAGGAGTTATAATGGTGCCTATGCCGCAAGGACGCAACAGA
GAAAGTGTTATACTTGACGTATCCGCAGAGATGACAGCAGATGATTTTATAATCGAT
TTCTTTGATGATAACAACATTGTCCATACGGAAAGAGGAGTTGGCCTATTTTCATTT
CCAATGTGTACCAGAATTAGATTTAGAGTTACACCATGGACACAACAAAAATCTCAG
AATGGACTTGACACTCCATCATTGGCTACGTGGGCGAACGGTACGTCTCCGAGGC
AGCCAGCGGTGTCTTTCATGTTTGAATTAAGAAGAACCTTCACTGAAAACGATTATA
AATTCGTTTCACGATGTACCTCGAAAGTTCAATATATATTGGATACCAACTTCCCAG
AGACATCATTTATTAACAGGCCTCAAATAGAATGGAACGTACAAGAGATGATTACTT
CTGACACAGACACAGTATGGTCACGTAAAATCGCAATGCTAGTCGCAGCATTTGCT
GCTAAGATCTGATTCTCC (SEQ ID NO: 13).
RVB/Horse-wt/USA/KY1-15/2021:
TGCGTCAGATTGCAGAAAAGAGTATTATCACTAGCTCCAAACACAAACTTGAACACT
GCAGGTCAGTCAATTCTCAATGATTATAATGCTATAGCATCCAGAGTGAATGGGAAG
ACTTATGCTCTTTTGGACCAAACAGCAATATTATCCCCTTACACGATACACGCACCG
ATAATTTCGCTGGCCGTTAGAATATCTACTGATGATTATGATGACATGAGAAATGGA
GTTGAGTCTATACTAGATTGTTTGGCTGCGGCGATTCGCACTGAAGGCTCGAGACC
GGTTAGAGTGATTGAACGTAGAGTTATTGAACCAGTGGTAAAGCAGCTGGTCGAAG
ATCTGAAGTTAAAAAGTCTGATTTCTGAAATCTCAATTGCCAATTTCGCTGCTACTGA
TACCGCGCTTATCCAACCAGAAGTAGTAGAAACTGAAAATCCATTGATAGTTGGTAT
CATAGAACAGGTGGTTGTAAGACAACCAGCCAGTCTAAATGGTGGCAATATTAGAG
CAGCGATTGGCAGATGGTCAGGTAATAAAGGCTCAGTCACATGTGTCTCAGGCATG
GAAGCAGAACATATGTTCTTCGTGGAACTAAAAGCTAGGACGTGTGGTGTACTGAA
CGTCGTTTATCTGCCAGCCCCAGGAGTTATAATGGTGCCTATGCCGCAAGGACGCA
ACAGAGAAAGTGTTATACTTGACGTATCCGCAGAGATGACAGCAGATGATTTTATAA
TCGATTTCTTTGATGATAACAACATTGTCCATACGGAAAGAGGAGTTGGCCTATTTT
CATTTCCAATGTGTACCAGAATTAGATTTAGAGTTACACCATGGACACAACAAAAAT
CTCAGAATGGACTTGACACTCCATCATTGGCTACGTGGGCGAACGGTACGTCTCCG
AGGCAGCCAGCGGTGTCTTTCATGTTTGAATTAAGAAGAACCTTCACTGAAAACGAT
TATAAATTCGTTTCACGATGTACCTCGAAAGTTCAATATATATTGGATACCAACTTCC
CAGAGACATCATTTATTAACAGGCCTCAAATAGAATGGAACGTACAAGAGATGATTA
CTTCTGACACAGACACAGTATGGTCACGTAAAATCGCAATGCTAGTCGCAGCATTT
GCTGCTAAGATCTGA (SEQ ID NO: 14).
RVB/Horse-wt/USA/KY1-16/2021:
TGCGTCAGATTGCAGAAAAGAGTATTATCACTAGCTCCAAACACAAACTTGAACACT
GCAGGTCAGTCAATTCTCAATGATTATAATGCTATAGCATCCAGAGTGAATGGGAAG
ACTTATGCTCTTTTGGACCAAACAGCAATATTATCCCCTTACACGATACACGCACCG
ATAATTTCGCTGGCCGTTAGAATATCTACTGATGATTATGATGACATGAGAAATGGA
GTTGAGTCTATACTAGATTGTTTGGCTGCGGCGATTCGCACTGAAGGCTCGAGACC
GGTTAGAGTGATTGAACGTAGAGTTATTGAACCAGTGGTAAAGCAGCTGGTCGAAG
ATCTGAAGTTAAAAAGTCTGATTTCTGAAATCTCAATTGCCAATTTCGCTGCTACTGA
TACCGCGCTTATCCAACCAGAAGTAGTAGAAACTGAAAATCCATTGATAGTTGGTAT
CATAGAACAGGTGGTTGTAAGACAACCAGCCAGTCTAAATGGTGGCAATATTAGAG
CAGCGATTGGCAGATGGTCAGGTAATAAAGGCTCAGTCACATGTGTCTCAGGCATG
GAAGCAGAACATATGTTCTTCGTGGAACTAAAAGCTAGGACGTGTGGTGTACTGAA
CGTCGTTTATCTGCCAGCCCCAGGAGTTATAATGGTGCCTATGCCGCAAGGACGCA
ACAGAGAAAGTGTTATACTTGACGTATCCGCAGAGATGACAGCAGATGATTTTATAA
TCGATTTCTTTGATGATAACAACATTGTCCATACGGAAAGAGGAGTTGGCCTATTTT
CATTTCCAATGTGTACCAGAATTAGATTTAGAGTTACACCATGGACACAACAAAAAT
CTCAGAATGGACTTGACACTCCATCATTGGCTACGTGGGCGAACGGTACGTCTCCG
AGGCAGCCAGCGGTGTCTTTCATGTTTGAATTAAGAAGAACCTTCACTGAAAACGAT
TATAAATTCGTTTCACGATGTACCTCGAAAGTTCAATATATATTGGATACCAACTTCC
CAGAGACATCATTTATTAACAGGCCTCAAATAGAATGGAACGTACAAGAGATGATTA
CTTCTGACACAGACACAGTATGGTCACGTAAAATCGCAATGCTAGTCGCAGCATTT
GCTGCTAAGATCTGATTC (SEQ ID NO: 15).
Reference Strain, MZ327693.1:
GTGTACGAGCATGGATCTGATCGAAACGGTGAACGCTTGCGTCAGATTGCAGAAAA
GAGTATTATCACTAGCTCCAAACACAAACTTGAACACTGCAGGTCAGTCAATTCTCA
ATGATTATAATGCTATAGCATCCAGAGTGAATGGGAAGACTTATGCTCTTTTGGACC
AAACAGCAATATTATCCCCTTACACGATACACGCACCGATAATTTCGCTGGCCGTTA
GAATATCTACTGATGATTATGATGACATGAGAAATGGAGTTGAGTCTATACTAGATT
GTTTGGCTGCGGCGATTCGCACTGAAGGCTCGAGACCGGTTAGAGTGATTGAACG
TAGAGTTATTGAACCAGTGGTAAAGCAGCTGGTCGAAGATCTGAAGTTAAAAAGTCT
GATTTCTGAAATCTCAATTGCCAATTTCGCTGCTACTGATACCGCGCTTATCCAACC
AGAAGTAGTAGAAACTGAAAATCCATTGATAGTTGGTATCATAGAACAGGTGGTTGT
AAGACAACCAGCCAGTCTAAATGGTGGCAATATTAGAGCAGCGATTGGCAGATGGT
CAGGTAATAAAGGCTCAGTCACATGTGTCTCAGGCATGGAAGCAGAACATATGTTC
TTCGTGGAACTAAAAGCTAGGACGTGTGGTGTACTGAACGTCGTTTATCTGCCAGC
CCCAGGAGTTATAATGGTGCCTATGCCGCAAGGACGCAACAGAGAAAGTGTTATAC
TTGACGTATCCGCAGAGATGACAGCAGATGATTTTATAATCGATTTCTTTGATGATA
ACAACATTGTCCATACGGAAAGAGGAGTTGGCCTATTTTCATTTCCAATGTGTACCA
GAATTAGATTTAGAGTTACACCATGGACACAACAAAAATCTCAGAATGGACTTGACA
CTCCATCATTGGCTACGTGGGCGAACGGTACGTCTCCGAGGCAGCCAGCGGTGTC
TTTCATGTTTGAATTAAGAAGAACCTTCACTGAAAACGATTATAAATTCGTTTCACGA
TGTACCTCGAAAGTTCAATACATATTGGAGACCAACTTCCCAGAGACATCATTTATT
AACAGGCCTCAAATAGAATGGAACGTACAAGAGATGATTACTTCTGACACAGACAC
AGTATGGTCACGTAAAATCGCAATGCTAGTCGCAGCATTTGCTGCTAAGATCTGATT
CTCCCTGAGCCCGGGAGCCGGGTTGCTCTAGAG (SEQ ID NO: 16).
MDLIETVNACVRLQKRVLSLAPNTNLNTAGQSILNDYNAIASRVNGKTYALLDQT
AILSPYTIHAPIISLAVRISTDDYDDMRNGVESILDCLAAAIRTEGSRPVRVIERRVIEPVVK
QLVEDLKLKSLISEISIANFAATDTALIQPEVVETENPLIVGIIEQVVVRQPASLNGGNIRAA
IGRWSGNKGSVTCVSGMEAEHMFFVELKARTCGVLNVVYLPAPGVIMVPMPQGRNRE
SVILDVSAEMTADDFIIDFFDDNNIVHTERGVGLFSFPMCTRIRFRVTPWTQQKSQNGL
DTPSLATWANGTSPRQPAVSFMFELRRTFTENDYKFVSRCTSKVQYILETNFPETSFIN
RPQIEWNVQEMITSDTDTVWSRKIAMLVAAFAAKI (SEQ ID NO: 17).

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Cell lines and viruses: MA-104 cells (ATCC® CRL-2378.1™, American Type Culture Collection [ATCC], Manassas, VA, USA) were maintained as previously described (Carossino M, et al. Virus Res. 2018, 255:39-54). Tissue culture fluid (TCF) derived from MA-104 cells infected with ERVA strain H2 (G3P[12]), ERVA strains RVA/Horse-tc/ARG/E8701-5MCCH/2016/G14P[12], RVA/Horse-tc/ARG/E8701-6MCBI/2016/G14P[12] and RVA/Horse-tc/ARG/E8701-9MCGR/2016/G14P[12]; bovine RVA (BRVA) strain NCDV-Lincoln, BRVA strain B223 porcine RVA (PRVA) strain OSU, PRVA strain Gottfried, simian RVA strain SA11, human RVA strain Wa (TC-adapted; ATCC VR-2018), human RVA strain Hu/Australia/1-9-12/77/S (ATCC VR-1546), and RVA reassortant WI79-4 (ATCC VR-2377) were used to assess specificity of the developed RT-qPCR assays as previously described (Carossino M, et al. Virus Res. 2018, 255:39-54).

Viral RNA and bacterial DNA: RNA and DNA from the following viruses and bacteria that cause diarrhea in horses were included for specificity evaluation of the newly developed 4-plex RT-qPCR assay: TCF containing ERVA strains RVA/Horse-tc/GBR/H2/1976/G3P[12], RVA/Horse-tc/ARG/E8701-5MCCH/2016/G14P[12], RVA/Horse-tc/ARG/E8701-6MCBI/2016/G14P[12] and RVA/Horse-tc/ARG/E8701-9MCGR/2016/G14P[12] (Carossino M, et al. Virus Res. 2018, 255:39-54); TCF containing equine coronavirus strain NC99 (Zhang J, et al. Virology. 2007, 369(1):92-104), and TCF containing equine rhinitis A (NVSL-0600EDV8501) and B (NVSL-0610EDV85010) viruses.

Fecal samples: A total of 193 fecal samples from diarrheic foals were included in this study. From these, 128 were either collected from farms in Central Kentucky during the 2017 foaling season (n=112 (Carossino M, et al. Virus Res. 2018, 255:39-54)) or during an outbreak of diarrhea in 2021 (n=16), and 65 were derived from outbreaks in Argentina between 2009 and 2014 (Carossino M, et al. Virus Res. 2018, 255:39-54; Mino S, et al. J Equine Vet Sci. 2017, 59:64-70). Ten percent fecal suspensions in serum-free EMEM were prepared as previously described (Carossino M, et al. Virus Res. 2018, 255:39-54) and fecal suspensions were stored at −80° C.

Transmission electron microscopy (TEM): Fecal samples from the 2021 diarrhea outbreak (n=16) were diluted to 10% in deionized water and separated into two aliquots. The first aliquot was clarified by a 2 min centrifugation at 12,000 g and a formvar, carbon-coated, 400-mesh copper grid was floated on a 50 μl drop of the supernatant for 15 min. Excess supernatant was removed from the grid with filter paper, and stained with 3% aqueous phosphotungstic acid, pH 7.0, for 1 min. The second aliquot was first centrifuged at 1,000 g for 15 min and the supernatant was subsequently centrifuged at 10,000 g for 30 min and 40,000 g for 1 h. The supernatant was discarded and the pellet was resuspended in purified deionized water and a 50 μl drop of the resuspension was stained as indicated above. Grids were viewed with a JEOL JEM-1011 Transmission Electron Microscope at an accelerating voltage of 100 kV. Representative viral particles were digitally imaged using an XR80M Wide-Angle Multi-Discipline Mid-Mount CCD Camera from AMT (Advanced Microscopy Techniques).

Nucleic acid isolation: Nucleic acid isolation was performed using the Taco™ mini nucleic acid extraction system (GeneReach USA, Lexington, MA, USA) as previously described (Zhang J, et al. Virology. 2007, 369(1):92-104). Two hundred microliters of 10% fecal suspension or tissue culture supernatant were used, eluted in 200 μl of elution buffer and stored at −80° C.

RT-PCR amplification of ERVA VP7 (segment 9) and ERVB VP6 genes (segment 6) and Sanger sequencing for G-typing: ERVA VP7-specific (gene segment 9) and ERVB VP6-specific (gene segment 6) standard RT-PCR assay and sequencing were performed as previously described (Mino S, et al. J Gen Virol. 2016, 97(4):912-921), and used as the gold-standard method for ERVA and ERVB detection in fecal specimens (Garaicoechea L, et al. Vet Microbiol. 2011, 148(2-4):150-160; Chang K O, et al. Arch Virol. 1996, 141(9):1727-1739). A high-fidelity RT-PCR kit (Qiagen One-Step Ahead RT-PCR kit) was used for generating full-length amplicons of ERVA VP7 and partial amplicons of ERVB VP6 for sequencing as previously described (Carossino M, et al. Virus Res. 2018, 255:39-54). DNA was submitted for Sanger sequencing to a commercial company (Eurofins Genomics LLC, Louisville, KY, USA), and both DNA strands of ERVA VP7 or ERVB VP6 amplicons were sequenced using a panel of primers specified in Table 1.

Sequence analysis was performed using Geneious R7 (Biomatters Inc., Newark, NJ, USA). ERVA G-types were identified using an automated genotyping tool for RVA (RotaC 2.0) (Maes P, et al. BMC Microbiol. 2009, 9:238).

Primer and probe design: Previously developed ERVA-specific primers and probes with modified dyes were used in this study (Table 2). ERVB VP6 and NSP5-specific forward and reverse primers and probes were designed using the PrimerQuest tool (Table 2). The primer and probe sequences were checked for specificity using the NCBI Basic Local Alignment Search Tool (BLAST) while self-annealing sites, hairpin loop formation and 3′ complementarity were verified using the IDT OligoAnalyzer tool.

TABLE 1
Primers used for RT-PCR amplification and sequencing of VP7 (genome segment 9) of ERVA and VP6
(genome segment 6) of ERVB

| | | Nucleotide | | |
|---|---|---|---|---|
| Primer name | Target | Position | Sequence (5′ to 3′) | Application |
| RVAVP7-Gra-5 | ERVA VP7 | 1-20ª | GGCTTTAAAAGCGAGAATTT (SEQ ID NO: 18) | RT-PCR and |
| | | | | sequencing |
| RVAVP7-Gra-3 | ERVA VP7 | 1,062-1,044ª | GGTCACATCATACAACTCT (SEQ ID NO: 19) | RT-PCR and |
| | | | | sequencing |
| RVAVP7-389-R | ERVA VP7 | 389-370ª | CCAGTAGGCCATCCTTTAGT (SEQ ID NO: 20) | Sequencing |
| RVAVP7-635-F | ERVA VP7 | 635-659ª | GTCCACTTAATACACAAACTCTAGG (SEQ ID NO: 21) | Sequencing |
| RVAVP7-241-R | ERVA VP7 | 245-220ª | GCAGTRTCCATTGAACCAGTAATTG (SEQ ID NO: 22) | Sequencing |
| RVAVP7-852-F | ERVA VP7 | 856-879ª | GAYATAACGGCTGATCCAACTACG (SEQ ID NO: 23) | Sequencing |
| RVAVP7-881-F | ERVA VP7 | 885-906ª | CTCCACAGATTGGACGAATGA (SEQ ID NO: 24) | Sequencing |
| RVBVP6-29-F | ERVB VP6 | 29-48^{b} | GTGAACGCTTGCGTCAGATT (SEQ ID NO: 25) | RT-PCR and |
| | | | | sequencing |
| RVBVP6-1201-R | ERVB VP6 | 1,201-1,182^{b} | CGGGCTCAGGGAGAATCAGA (SEQ ID NO: 26) | RT-PCR and |
| | | | | sequencing |
| RVBVP6-744-F | ERVB VP6 | 744-763^{b} | CCGCAGAGATGACAGCAGAT (SEQ ID NO: 27) | Sequencing |
| RVBVP6-455-R | ERVB VP6 | 455-436^{b} | CTGGTTGGATAAGCGCGGTA (SEQ ID NO: 28) | Sequencing |

^{a}nucleotide position based on GenBank Accession number KM454508.1
^{b}nucleotide position based on GenBank Accession number MZ327693.1

TABLE 2
Primers and probe combinations for the detection of rotavirus A (pan-rotavirus A, targeting
the NSP3 gene), VP7 gene of ERVA G3 and G14 genotypes, VP6 of ERVB and NSP5 of ERVB

| | | Nucleotide | |
|---|---|---|---|
| Name | Target | Position | Sequence (5′ to 3′) |
| NVP3-FDeg¹ | RVA NSP3 | 963-982ª | ACCATCTWCACRTRACCCTC (SEQ ID NO: 29) |
| NVP3-R1¹ | RVA NSP3 | 1,053-1,034ª | GGTCACATAACGCCCCTATA (SEQ ID NO: 30) |
| NVP3-Probe¹ | RVA NSP3 | 984-1,026ª | JUN-ATGAGCACAATAGTTAAAAGCTAACACTGTCAA-QSY (SEQ |
| | | | ID NO: 31) |
| RVA-G3-756F | ERVA VP7 (G3)^{b} | 756-777 | GATGTTACCACGACCACTTGTA (SEQ ID NO: 32) |
| RVA-G3-872R | ERVA VP7 (G3)^{b} | 872-854 | AGTTGGATCGGCCGTTATG (SEQ ID NO: 33) |
| RVA-G3-779P | ERVA VP7 (G3)^{b} | 779-823 | FAM-TGGGACCACGAGAGAATGTAGCTGT-MGB (SEQ ID NO: 34) |
| RVA-G14-ARG869F | ERVA VP7 (G14)^{c} | 869-885 | ATCCGACTACGGCTCCA (SEQ ID NO: 35) |
| RVA-G14-ARG1011R | ERVA VP7 (G14)^{c} | 1,011-990 | TGCAGCAGAATTTAATGATCGC (SEQ ID NO: 36) |
| RVA-G14-ARG886P | ERVA VP7 (G14)^{c} | 886-915 | VIC-CAGATTGGACGAATGATGCGTATAAATTGG-MGB (SEQ ID |
| | | | NO: 37) |
| ERVB-VP6-F | ERVB VP6 | 132-153^{d} | CATCCAGAGTGAATGGGAAGAC (SEQ ID NO: 38) |
| ERVB-VP6-R | ERVB VP6 | 230-210^{d} | TTCTAACGGCCAGCGAAATTA (SEQ ID NO: 39) |
| ERVB-VP6-P | ERVB VP6 | 187-209^{d} | LIZ-CCCTTACACGATACACGCACCGA-QSY (SEQ ID NO: 40) |
| ERVB-NSP5-F | ERVB NSP5 | 124-146^{e} | GCCTTCTGATTCTACGTCAACTA (SEQ ID NO: 41) |
| ERVB-NSP5-R | ERVB NSP5 | 238-215^{e} | CTTGTTGTACGCTTCTTCGTATTC (SEQ ID NO: 42) |
| ERVB-NSP5-P | ERVB NSP5 | 160-183^{e} | LIZ-AACATCAAGTCGTAGCGACGCAGT-QSY (SEQ ID NO: 43) |

¹Primers and probe name and sequences derived from Freeman et al., 2008. W = T, U, A; R = A, G.
^{a}nucleotide position based on GenBank Accession number X81436
^{b}nucleotide position based on GenBank Accession number KM454497.1
^{c}nucleotide position based on GenBank Accession number KM454508.1
^{d}nucleotide position based on GenBank Accession number MZ327693.1
^{e}nucleotide position based on GenBank Accession number MZ327698.1
FAM, 6-carboxyfluorescein; JUN, JUN ™ dye; LIZ, LIZ ™ dye; MGB, minor groove binder; QSY, QSY ™ quencher; VIC, VIC ™ dye.

Synthesis of ERVA and ERVB in vitro transcribed RNA for analytical performance evaluation: For ERVA, a previously synthesized in vitro transcribed (IVT) RNA with a 493 nt insert containing the targeted regions (NSP3, G3 VP7 and G14 VP7) was prepared and used as described (Carossino M, et al. Virol J. 2019, 16(1):49). A similar approach was used to develop ERVB IVT RNA containing the target sequences. Briefly, a 214 bp insert containing the target regions (VP6 [nt position 132-230] and NSP5 [nt position 124-238] from ERVB strain Rotavirus B isolate RVB/Horse-wt/USA/KY1518/2021 (GenBank Accession numbers KM454497.1 and KM454508.1, respectively) was cloned into the pGEM-3Z vector (Promega, Madison, WI) downstream of the T7 promoter (pERVBVP6NSP5) by GeneArt Gene Synthesis (ThermoFisher Scientific, Regensburg, Germany). Transformed E. coli K12 DH10B™ T1R were cultured overnight at 37° C. with shaking (270 rpm), plasmid DNA was purified, linearized with HindII and subjected to in vitro transcription using the Megascript T7 Transcription kit (ThermoFisher Scientific, Waltham, MA) followed by DNase treatment and purification as we previously described in detail (Carossino M, et al. Virol J. 2019, 16(1):49). The number of ERVA and ERVB IVT RNA molecules per microliter (copies/μl) was calculated according to the following formula: Number of IVT RNA molecules/L

$$Number of I V T R N A molecules / μL = \frac{Avogadro ' s number ( 6.022 \times 1 {0}^{23} ) \times I V T R N A concentration ( \text{g/} μL )}{I V T R N A molecular weight ( g )}$$

The concentration of IVT RNA was adjusted to 10⁷ copies/μl using nuclease-free water containing 40 ng/μl of Ambion® Yeast tRNA (ThermoFisher Scientific), and serially ten-fold diluted (10⁷-1 IVT RNA copies/μl) using nuclease-free water containing Ambion® Yeast tRNA.

Analysis of fecal samples: A total of 193 fecal samples were included in the study, from which 177 were archived samples used in previous studies (Carossino M, et al. Virus Res. 2018, 255:39-54; Carossino M, et al. Virol J. 2019, 16(1):49) and 16 were derived from a recent (2021 foaling season) outbreak of diarrhea in foals from Central Kentucky during which ERVB was first identified.

From the 193 samples, 93 samples were confirmed negative for ERVA and ERVB, 85 were positive for ERVA as determined by VP7-specific standard RT-PCR (Carossino M, et al. Virus Res. 2018, 255:39-54; Mino S, et al. J Equine Vet Sci. 2017, 59:64-70) and 15 (derived from the 2021 foaling season) were positive for ERVB by VP6-specific standard RT-PCR. From the 85 ERVA-positive samples, 41 and 44 were confirmed as G3 or G14 genotype by sequencing of the VP7 gene, respectively.

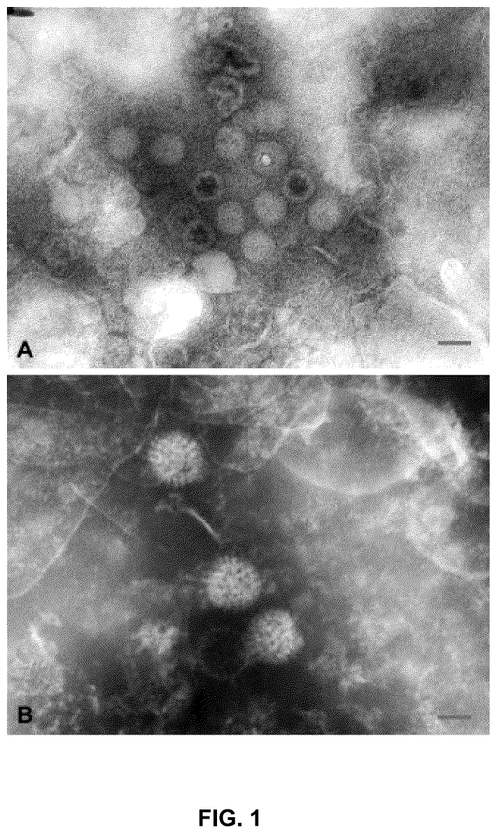

The ERVB-positive samples derived from the 2021 foaling season (n=15) were subjected to TEM. Rotaviral particles were evident in a total of seven samples (1 through 5, 9, and 13, as shown in FIG. 1.

Analytical performance of ERVA and ERVB-specific multiplex RT-qPCR assays targeting ERVA NSP3, G3 VP7, and G14 VP7 together with either ERVB VP6 or NSP5.:

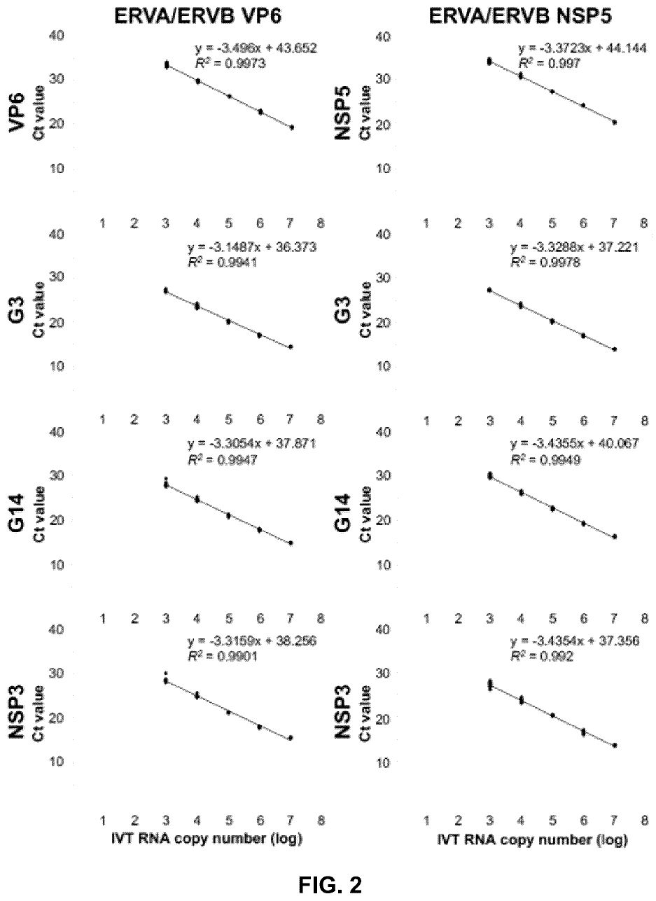

(i) Analytical sensitivity and specificity of ERVA/ERVB-VP6-specific multiplex RT-qPCR assay: The analytical sensitivity of the ERVA/ERVB-VP6-specific multiplex RT-qPCR assays was determined using a ten-fold serial dilution series (6-12 replicates per dilution) of IVT RNA (10⁷ to 1 IVT RNA copies/μl) containing the target sequences. Standard curves were generated for each of the four targets on the linear range (G3 VP7, G14 VP7, ERVA NSP3 and ERVB VP6). Performance parameters are summarized in Table 3. Perfect linearity (R²>0.99, Table 3 and FIG. 2) and amplification efficiencies of 108%, 100%, 100% and 93%, respectively, were confirmed. The LOD was determined to be 100 and 1,000 copies/μl of IVT RNA for the three ERVA targets and ERVB VP6, respectively. Compared to the singleplex ERVB VP6-specific assay, there is a 10-fold difference in the detection rate (Table 3).

The ERVA/ERVB VP6 4-plex assay proved to be specific for detection of group A rotaviruses of various animal species and human (via the RVA NSP3 target, serving as a pan-group A rotavirus assay), as well as specific for the respective ERVA genotypes G3 and G14 and ERVB targets and did not amplify other viruses or bacteria associated with diarrhea in horses. The ERVA genotyping targets (G3 and G14 VP7) performed as previously reported, with no cross-reactivity between each other. No cross-reactivity between ERVA and ERVB detection was noted.

(ii) Analytical sensitivity and specificity of ERVA/ERVB-NSP5-specific multiplex RT-qPCR assay: The analytical sensitivity of the ERVA/ERVB-NSP5-specific multiplex RT-qPCR assay was determined as described for the ERVA/ERVB VP6 4-plex assay and results are summarized in Table 3. This assay also demonstrated perfect linearity (R²>0.99, Table 3 and FIG. 2) and equivalent LOD, but amplification efficiencies were overall lower across targets compared to the ERVA/ERVB VP6 4-plex assay (Table 3). Similarly to the ERVB VP6-specific singleplex assay, there is a 10-fold difference in the detection rate between the singleplex ERVB NSP5-specific assay and the ERVA/ERVB NSP5 4-plex assay (Table 3). Similarly to the ERVB VP6-specific singleplex assay, there is a 10-fold difference in the detection rate between the singleplex ERVB NSP5-specific assay and the ERVA/ERVB NSP5 4-plex assay (Table 3). The assay's specificity was equal to that of the ERVA/ERVB VP6 4-plex assay, and no off-target amplification was noted.

(iii) Precision assessment of ERVA/ERVB VP6 and ERVA/ERVB NSP5-specific multiplex RT-qPCR assays. To determine assays' precision, both within-run and between-run imprecision were determined. In all cases, CV was less than 3%, indicating that both assays have high repeatability (within-run) and reproducibility (between-run) (Table 4).

TABLE 3
Analytical performance of multiplex RT-qPCR assays for
the detection and genotyping of equine rotavirus A
and detection of equine rotavirus B (VP6 or NSP5)

| | 4-plex (ERVA/ERVB VP6) | | | |
|---|---|---|---|---|
| Parameter | G3 | G14 | NSP3 | VP6 |
| Slope | −3.1487 | −3.3054 | −3.3159 | −3.496 |
| Linearity (R²) | >0.99 | >0.99 | >0.99 | >0.99 |
| Efficiency (%) | 108 | 100 | 100 | 93.22 |
| LOD_{95%} (copies/μl) | 67 | 67 | 67 | 747 |
| Detection rate limit | 100 | 100 | 100 | 1000 |
| (100%, copies/μl) | | | | |
| Ct cut-off | 34 | 39 | 35 | 34 |
| | 4-plex (ERVA/ERVB NSP5) | | | |
| Parameter | G3 | G14 | NSP3 | NSP5 |
| Slope | −3.3288 | −3.4355 | −3.4354 | −3.3723 |
| Linearity (R²) | >0.99 | >0.99 | >0.99 | >0.99 |
| Efficiency (%) | 100 | 95 | 95 | 98 |
| LOD_{95%} (copies/μl) | 67 | 67 | 67 | 747 |
| Detection rate limit | 100 | 100 | 100 | 1000 |
| (100%, copies/μl) | | | | |
| Ct cut-off | 35 | 36 | 34 | 35 |

LOD_{95%}, limit of detection 95%; Ct, cycle threshold

TABLE 4
Precision evaluation of the ERVA/ERVB VP6-specific and ERVA/ERVB NSP5-
specific multiplex RT-qPCR assays. a) Within-run and b) between-run
imprecision. Values represent coefficient of variation in %

| | a) Within-run | | | | | | | |
|---|---|---|---|---|---|---|---|---|
| Concentration of target | ERVA/ERVB VP6 4-plex assay | | | | ERVA/ERVB NSP5 4-plex assay | | | |
| (IVT RNA copies/μl) | G3 | G14 | NSP3 | VP6 | G3 | G14 | NSP3 | NSP5 |
| 100,000 | 0.55% | 0.43% | 0.66% | 0.33% | 0.86% | 0.91% | 0.77% | 0.51% |
| 10,000 | 1.62% | 1.05% | 1.11% | 0.53% | 1.11% | 0.85% | 1.91% | 0.97% |
| 1,000 | 0.89% | 1.68% | 1.87% | 1.11% | 0.51% | 1.3% | 1.99% | 0.99% |
| | b) Between-run | | | | | | | |
| Concentration of target | ERVA/ERVB VP6 4-plex assay | | | | ERVA/ERVB NSP5 4-plex assay | | | |
| (IVT RNA copies/μl) | G3 | G14 | NSP3 | VP6 | G3 | G14 | NSP3 | NSP5 |
| 100,000 | 1.2% | 1.1% | 0.30% | 0.32% | 1.2% | 1.1% | 0.66% | 0.30% |
| 10,000 | 0.95% | 1.23% | 1.35% | 0.45% | 1.05% | 1.14% | 1.77% | 0.80% |
| 1,000 | 1.07% | 2.31% | 2.17% | 0.90% | 0.46% | 0.93% | 1.03% | 0.59% |

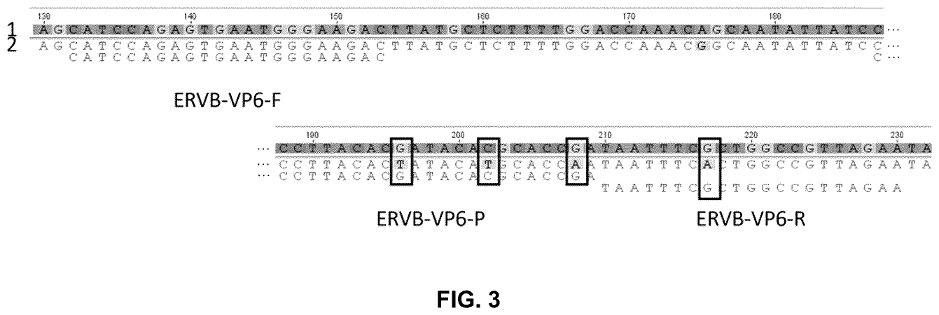

Clinical performance of the ERVA/ERVB VP6-specific multiplex RT-qPCR assay targeting ERVA NSP3, G3 VP7, G14 VP7 and ERVB VP6 genes. Based on the overall higher analytical efficiency across targets of the ERVA/ERVB VP6 4-plex assay compared to that of the ERVA/ERVB NSP5 4-plex assay (Table 3), the former was selected for further evaluation of its clinical performance using a total of 193 fecal samples. Overall, the new ERVA/ERVB VP6 4-plex assay correctly identified most of the fecal samples with only few exceptions and a high level of agreement compared to RT-PCR (96.4-99.5% and kappa 0.926-0.985). The specificity for all targets in the ERVA/ERVB VP6 4-plex assay was 100% compared to RT-PCR, with no non-specific amplifications observed in negative samples. The NSP3 (pan-RVA) assay showed a sensitivity of 91.8% when compared to RT-PCR. In the case of the G3 and G14 VP7 targets, the ERVA/ERVB VP6 4-plex assay was able to correctly genotype 38/41 ERVA G3 samples and 44/45 ERVA G14 samples when compared to RT-PCR and Sanger sequencing, yielding a sensitivity of 92.7% and 97.8%, respectively. Regarding detection of ERVB, the ERVA/ERVB VP6 4-plex assay was able to correctly detect ERVB in 14/15 samples (sensitivity of 93.3%). Two of the positive ERVB samples (RVB/Horse-wt/USA/KY1-6/2021 and RVB/Horse-wt/USA/KY1-13/2021) showed approximately a 4% difference in their nucleotide sequence compared to the VP6 of the reference strain (GenBank Accession Number MZ327693.1), which included a total of 45 and 47 nucleotide substitutions for RVB/Horse-wt/USA/KY1-6/2021 and RVB/Horse-wt/USA/KY1-13/2021, respectively. Among these, three and one nucleotide substitutions were located in the probe (ERVB-VP6-P) and reverse primer (ERVB-VP6-R) sequences, respectively (G₁₉₆→T₁₉₆; C₂₀₂→T₂₀₂; G₂₀₈→A₂₀₃; G₂₁₇→A₂₁₇; FIG. 3). In spite of these differences, the ERVA/ERVB VP6 4-plex assay was able to readily detect RVB/Horse-wt/USA/KY1-13/2021, while RVB/Horse-wt/USA/KY1-6/2021 yielded undetermined results. Since the assay was still able to amplify one of these samples despite the nucleotide substitutions within the probe and reverse primer sequences, a sample-specific PCR inhibitor was suspected in this case.

Finally, we compared the sensitivity for each of the targets in common between this ERVA/ERVB VP6 4-plex assay and that of our previously described ERVA 3-plex assay. The NSP3 (pan-RVA) assay was the only ERVA-specific target which showed a slightly reduced sensitivity (91.8%, 7/85 positive samples that yielded either Ct values>35 [n=2] or undetermined results [n=5]) compared with the previously described ERVA 3-plex assay (p-value=0.0083) in which sensitivity was 100% (Carossino et al., 2019). For G3 and G14 targets, the sensitivities as determined with the ERVA/ERVB VP6 4-plex assay were statistically equivalent to those reported for the ERVA 3-plex assay (p-values>0.05) (Carossino et al., 2019). The specificities in all cases were 100%.

What is claimed:

1. A panel of oligonucleotides for use in a multiplex reverse transcriptase-polymerase chain reaction (RT-PCR) assay for the identification of rotavirus A and B and genotypes thereof, the panel of nucleotides comprising:
a rotavirus A-specific forward PCR primer, a rotavirus A-specific reverse PCR primer, and a labeled oligonucleotide probe, wherein the forward PCR primer, reverse PCR primer, and the labeled oligonucleotide probe are each complementary to a region of a nucleic acid, or the complement thereof, encoding the non-structural protein 3 (NSP3) of an equine rotavirus A;
a rotavirus A VP7 (subtype G3 genotype)-specific forward PCR primer, a rotavirus A VP7 (subtype G3 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof;
a rotavirus A VP7 (subtype G14 genotype)-specific forward PCR primer, a rotavirus A VP7 (subtype G14 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; and
either
(i) a rotavirus B VP6-specific forward PCR primer, a rotavirus B VP6-specific reverse PCR primer, and a labeled oligonucleotide, wherein the forward PCR primer, reverse PCR primer, and the labeled oligonucleotide probe are each complementary to a nucleic acid, or the complement thereof, encoding the structural protein VP6, or a fragment thereof, of an equine rotavirus B,
or
(ii) a rotavirus B non-structural protein 5 (NSP5)-specific forward PCR primer, a rotavirus B an NSP5-specific reverse PCR primer, and a labeled oligonucleotide probe, wherein the forward PCR primer, reverse PCR primer, and the labeled oligonucleotide probe are each complementary to a nucleic acid, or the complement thereof, encoding the non-structural protein NSP5, or a fragment thereof, of an equine rotavirus B.

2. The panel of claim 1, wherein the primers and the labeled oligonucleotide probes are selected from the group consisting of:
NVP3-FDeg,
(SEQ ID NO: 29)
ACCATCTWCACRTRACCCTC;
NVP3-R1,
(SEQ ID NO: 30)
GGTCACATAACGCCCCTATA;
NVP3-Probe,
(SEQ ID NO: 31)
JUN-ATGAGCACAATAGTTAAAAGCTAACACTGTCAA-QSY;
RVA-G3-756F,
(SEQ ID NO: 32)
GATGTTACCACGACCACTTGTA;
RVA-G3-872R,
(SEQ ID NO: 33)
AGTTGGATCGGCCGTTATG;
RVA-G3-779P,
(SEQ ID NO: 34)
FAM-TGGGACCACGAGAGAATGTAGCTGT-MGB;
RVA-G14-ARG869F,
(SEQ ID NO: 35)
ATCCGACTACGGCTCCA;
RVA-G14-ARG1011R,
(SEQ ID NO: 36)
TGCAGCAGAATTTAATGATCGC;
RVA-G14-ARG886P,
(SEQ ID NO: 37)
VIC-CAGATTGGACGAATGATGCGTATAAATTGG-MGB;
ERVB-VP6-F,
(SEQ ID NO: 38)
CATCCAGAGTGAATGGGAAGAC;
ERVB-VP6-R,
(SEQ ID NO: 39)
TTCTAACGGCCAGCGAAATTA;
ERVB-VP6-P,
(SEQ ID NO: 40)
LIZ-CCCTTACACGATACACGCACCGA-QSY;
ERVB-NSP5-F,
(SEQ ID NO: 41)
GCCTTCTGATTCTACGTCAACTA;
ERVB-NSP5-R,
(SEQ ID NO: 42)
CTTGTTGTACGCTTCTTCGTATTC;
and
ERVB-NSP5-P,
(SEQ ID NO: 43)
LIZ-AACATCAAGTCGTAGCGACGCAGT-QSY,
wherein each of the labeled oligonucleotide probes has a detectable label conjugated at each of the 5′ and the 3′ termini of the oligonucleotide.

3. The panel of claim 2, wherein the detectable label conjugated at each of the 5′ and the 3′ termini of the oligonucleotide is selected from the group consisting of FAM, 6-carboxyfluorescein; JUN, JUN® dye; LIZ, LIZ® dye; MGB, minor groove binder; QSY, QSY® quencher; and VIC, VIC® dye.

4. An RT-PCR multiplex method for determining whether an equine are infected with an equine rotavirus, the method comprising the steps of:
(a) obtaining a fecal sample from the equine;
(b) extracting RNA from the fecal sample;
(c) assaying the RNA by an RT-PCR multiplex assay performed with a panel of nucleotides comprising:
a rotavirus A-specific forward PCR primer, a rotavirus A-specific reverse PCR primer, and a labeled oligonucleotide probe, wherein the forward PCR primer, reverse PCR primer, and the labeled oligonucleotide probe are each complementary to a region of a nucleic acid, or the complement thereof, encoding the non-structural protein 3 (NSP3) of an equine rotavirus A;
a rotavirus A VP7 (subtype G3 genotype)-specific forward PCR primer, a rotavirus A VP7 (subtype G3 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof;
a rotavirus A VP7 (subtype G14 genotype)-specific forward PCR primer, a rotavirus A VP7 (subtype G14 genotype)-specific reverse PCR primer, and a labeled oligonucleotide probe, wherein the forward PCR primer, reverse PCR primer, and the labeled oligonucleotide probe are each complementary to a nucleic acid, or the complement thereof, encoding the structural protein VP7 of an equine rotavirus A, or a fragment thereof; and
either
(i) a rotavirus B VP6-specific forward PCR primer, a rotavirus B VP6-specific reverse PCR primer, and a labeled oligonucleotide, wherein the forward PCR primer, reverse PCR primer, and the labeled oligonucleotide probe are each complementary to a nucleic acid, or the complement thereof, encoding the structural protein VP6, or a fragment thereof, of an equine rotavirus B,
or
(ii) a rotavirus B non-structural protein 5 (NSP5)-specific forward PCR primer, a rotavirus B an NSP5-specific reverse PCR primer, and a labeled oligonucleotide probe, wherein the forward PCR primer, reverse PCR primer, and the labeled oligonucleotide probe are each complementary to a nucleic acid, or the complement thereof, encoding the non-structural protein NSP5, or a fragment thereof, of an equine rotavirus B,
wherein detection of the rotavirus A or B indicates an infection of the equine with the rotavirus A or B and whether, if present, the rotavirus A is of the genotype VP7 subtype G3 or G14.

5. The method of claim 4, wherein the primers and the labeled oligonucleotide probes are selected from the group consisting of:
NVP3-FDeg,
(SEQ ID NO: 29)
ACCATCTWCACRTRACCCTC;
NVP3-R1,
(SEQ ID NO: 30)
GGTCACATAACGCCCCTATA;
NVP3-Probe,
(SEQ ID NO: 31)
JUN-ATGAGCACAATAGTTAAAAGCTAACACTGTCAA-QSY;
RVA-G3-756F,
(SEQ ID NO: 32)
GATGTTACCACGACCACTTGTA;
RVA-G3-872R,
(SEQ ID NO: 33)
AGTTGGATCGGCCGTTATG;
RVA-G3-779P,
(SEQ ID NO: 34)
FAM-TGGGACCACGAGAGAATGTAGCTGT-MGB;
RVA-G14-ARG869F,
(SEQ ID NO: 35)
ATCCGACTACGGCTCCA;
RVA-G14-ARG1011R,
(SEQ ID NO: 36)
TGCAGCAGAATTTAATGATCGC;
RVA-G14-ARG886P,
(SEQ ID NO: 37)
VIC-CAGATTGGACGAATGATGCGTATAAATTGG-MGB;
ERVB-VP6-F,
(SEQ ID NO: 38)
CATCCAGAGTGAATGGGAAGAC;
ERVB-VP6-R,
(SEQ ID NO: 39)
TTCTAACGGCCAGCGAAATTA;
ERVB-VP6-P,
(SEQ ID NO: 40)
LIZ-CCCTTACACGATACACGCACCGA-QSY;
ERVB-NSP5-F,
(SEQ ID NO: 41)
GCCTTCTGATTCTACGTCAACTA;
ERVB-NSP5-R,
(SEQ ID NO: 42)
CTTGTTGTACGCTTCTTCGTATTC;
and
ERVB-NSP5-P,
(SEQ ID NO: 43)
LIZ-AACATCAAGTCGTAGCGACGCAGT-QSY,
wherein, each of the labeled oligonucleotide probes has a detectable label conjugated at each of the 5′ and the 3′ termini of the oligonucleotide, and
wherein detection of the rotavirus A or B indicates an infection of the equine with the rotavirus A or B and whether, if present, the rotavirus A is of the genotype VP7 subtype G3 or G14.
